(19) United States
(12) Patent Application Publication
(10) Pub. No.: US 2024/0008995 A1
(43) Pub. Date: Jan. 11, 2024

(54) GLENOID IMPLANTS

(71) Applicant: HOWMEDICA OSTEONICS CORP., Mahwah, NJ (US)

(72) Inventors: Shawn Martin GARGAC, Fort Wayne, IN (US); Alexander Paul WOLFE, Fort Wayne, IN (US); Austin Wyatt MUTCHLER, Warsaw, IN (US); Robert COURTNEY, Pierceton, IN (US); Alan IMM, Edgerton, OH (US); Benjamin DASSONVILLE, Saint Hilaire du Touvet (FR); Gilles HENRY, Le Pont de Claix (FR); David R. STUMP, Columbia City, IN (US); Vincent COULANGE, Lyon (FR); Vincent GABORIT, Saint Martin d'Hères (FR)

(73) Assignee: HOWMEDICA OSTEONICS CORP., Mahwah, NJ (US)

(21) Appl. No.: 18/253,612

(22) PCT Filed: Sep. 30, 2021
(86) PCT No.: PCT/US2021/071653

§ 371 Date: May 19, 2023

Related U.S. Application Data
(60) Provisional application No. 63/132,600, filed on Dec. 31, 2020.

(51) Int. Cl. A61F 2/40

(52) U.S. Cl. CPC A61F 2/4081; A61F 2002/4085

(57) ABSTRACT

Various embodiments of novel glenoid implant for replacing a portion of an articulation surface of a joint that provide various enhancements for glenoid implants is disclosed. Some examples of enhancements are improved quality of the primary fixation of glenoid implants and improved revisability of glenoid implants.

### FIELD OF DISCLOSURE

The present disclosure generally relates to glenoid implants for shoulder prosthesis.

### BACKGROUND

A shoulder prosthesis includes a glenoid implant intended to replace the glenoid cavity of the scapula and/or a humeral implant intended to replace the humeral head. The glenoid implant generally includes and articular body intended to articulate with the humeral head, and a fixation means to stabilize the articular body with respect to the scapula.

Optimum glenoid constraint may not be able to be achieved with a simple spherical surface. This principle is emphasized by the natural glenoid/labrum combination, which is not spherical and does not provide the same maximum constraint in all translation directions. Referring to FIG. 1, a currently available shoulder prosthesis glenoid component 10 has an articulation surface 12, which is essentially defined by a spherical or dual radius, fully concave geometry. As such, prior art glenoid components do not take into account the differing levels of constraint required for different activities or the varying curvature of the natural glenoid.

Moreover, since currently available glenoid components have fully concave articulation surfaces, as the humeral head translates, the contact point between the head and glenoid will approach the edge of the glenoid. At a certain point, as illustrated in FIG. 2, a load vector 20 being applied to the glenoid component 10 by a humeral head 22 will no longer pass through the glenoid 24, but will load the glenoid component 10 in an overhanging manner, significantly increasing loosening tendencies of the glenoid component 10.

Typically, the glenoid prosthetic components are provided with one or more pegs or one or more keels on the side opposite from the articulation surface 12. The pegs or keels are inserted into mating holes prepared in the glenoid cavity of the scapular neck. The pegs or keels are affixed to the scapular neck using bone cement.

Many prior art glenoid components are onlay design. In recent years, studies have found that inlay designs may offer improved stability of the glenoid component and reduce glenoid component loosening that are common in total shoulder arthroplasty. With growing interest in inlay designs for glenoid components, there is a need for new and improved inlay and/or onlay glenoid components.

### SUMMARY

Provided herein are various embodiments of glenoid implants that provide a replacement articulation surface of a glenoid in a shoulder. Many of the embodiments of the disclosed glenoid implants are inlay style implants with various fixation features. The implanted inlay implants will sit inside a reamed/drilled cavity in the glenoid such that the face opposing the articulation surface is positioned below the glenoid face. Some of the glenoid designs shown may be used as inlay or onlay implant.

### BRIEF DESCRIPTION OF THE DRAWINGS

The various embodiments of the inventive hydrogel implant of the present disclosure will be described in more detail in conjunction with the following drawing figures. The structures in the drawing figures are illustrated schematically and are not intended to show actual dimensions.

FIG. 1 is a perspective view of a prior art glenoid component with a spherical articulation surface and standard pegs.

FIG. 2 shows a side cross-sectional view of an illustration of an overhanging load on the prior art glenoid component.

FIG. 3 shows a glenoid prepared with a ring-shaped trough in which a ring-shaped glenoid implant according to the present disclosure is received.

FIG. 4 shows a fully seated ring-shaped glenoid implant of the present disclosure.

FIG. 5 shows a cross-sectional view of the fully seated ring-shaped glenoid implant.

FIG. 6 shows a cross-sectional view of the ring-shaped glenoid implant that is engaging a prosthetic humeral head.

FIG. 7 shows an embodiment of the ring-shaped glenoid implant comprising pegs along the bottom surface of the implant.

FIG. 8 shows another embodiment of the ring-shaped glenoid implant that is configured for cementless fixation into the glenoid.

FIG. 9 is a drill guide for identifying a center of a glenoid.

FIG. 10 is a perspective view of the drill guide of FIG. 9 positioned on a glenoid.

FIGS. 11-13 are illustrations showing the procedure for cutting a trough into the glenoid using a reamer.

FIG. 14 is an illustration of an example of a reamer for cutting a trough into the glenoid.

FIG. 15 is an illustration of a drill guide according to an embodiment of the present disclosure.

FIG. 16 is an illustration of a glenoid that has been prepared with a trough and blind holes for receiving the ring-shaped glenoid implant of FIG. 7.

FIG. 17 is an illustration of a glenoid implant according to another embodiment that is in an implanted state.

FIG. 18 is a side view illustration of the glenoid implant of FIG. 17.

FIG. 19 is an isometric view illustration showing the anchor surface of the glenoid implant of FIGS. 17-18.

FIG. 20-33 are illustrations showing the procedure for preparing a glenoid for the glenoid implant of FIGS. 17-19.

FIG. 34A is an isometric illustration of a glenoid implant according to another embodiment showing the articulation surface of the implant.

FIG. 34B is an isometric illustration of the glenoid implant of FIG. 34A showing the anchor surface of the implant.

FIGS. 35A-36B are a series of illustrations showing the procedure for preparing a glenoid for the glenoid implant of FIGS. 34A-34B.

FIGS. 37A-37B are illustrations showing a glenoid implant according to another embodiment.

FIG. 37C is a cross-sectional view of the glenoid implant of FIGS. 37A-37B.

FIGS. 37D-37F are illustrations showing an example of a procedure for preparing a glenoid to receive the glenoid implant of FIGS. 37A-37B.

FIGS. 37G-37I are illustrations showing a procedure for implanting the implant of FIGS. 37A-37B.

FIG. 38A-38B are illustrations showing a glenoid implant according to another embodiment.

FIG. 38C-38E are illustrations showing a procedure for implanting the implant of FIGS. 38A-38B into a prepared glenoid.

FIGS. 39A-39B are illustrations showing a glenoid implant according to another embodiment in its pre-implant configuration.

FIG. 39C is a cross-sectional view of the glenoid implant of FIGS. 39A-39B.

FIG. 39D-39E are illustrations showing the glenoid implant of FIGS. 39A-39B in its implanted configuration.

FIG. 39F is a cross-sectional view of the glenoid implant of FIGS. 39D-39E.

FIGS. 39G-39H are illustrations showing a procedure for preparing a glenoid to receive the glenoid implant of FIGS. 39D-39E.

FIG. 39I is a cross-sectional view illustration of a 2-in-1 reamer used in the procedure shown in FIGS. 39G-39H.

FIG. 39J is an illustration of the recess in a glenoid formed by the procedure shown in FIGS. 39G-39I.

FIGS. 39K-39P are illustrations showing a procedure for implanting the glenoid implant of FIGS. 39A-39B.

FIGS. 40A-40C are illustrations showing a glenoid implant according to another embodiment.

FIG. 40D is an illustration of a glenoid that is prepared with a recess to receive the glenoid implant of FIG. 40A.

FIG. 40E is an illustration of the glenoid implant of FIG. 40A seated in the glenoid.

FIGS. 40F-40G are illustrations of all-polymer glenoid implant according to another embodiment.

FIGS. 40H-40N, 40P, and 40Q are additional embodiments of the all-polymer glenoid implant.

FIG. 40R is an illustration of a glenoid that is prepared with a recess to receive the glenoid implant of FIG. 40J.

FIG. 40S is an illustration of the glenoid implant of FIG. 40J seated in the glenoid.

FIG. 40T is an illustration of the glenoid implant of FIG. 40I seated in the glenoid.

FIG. 40U is an illustration of a portion of the recess prepared in a glenoid to receive one of the all-polymer glenoid implants wherein the portion is configured to form an interference fit with the all-polymer glenoid implants.

FIGS. 41A-41F are illustrations of various embodiments of another glenoid implant according to the present disclosure.

FIG. 41G is a detailed illustration showing the interference fit between the glenoid and the glenoid implants shown in FIGS. 41A-41F.

FIGS. 42A-42D are illustrations of a metal-backed glenoid implant embodiment.

FIGS. 42E-42F are illustrations of the metal anchor component of the metal-backed glenoid implant of FIGS. 42A-42D.

FIGS. 42G-42H are illustrations of a glenoid prepared with a recess for receiving the metal-backed glenoid implant of FIGS. 42A-42C.

FIGS. 43A-43B are illustrations of a porous metal-backed glenoid implant embodiment.

FIG. 43C is an illustration of an exploded view of the porous metal-backed glenoid implant of FIGS. 43A-43B.

FIG. 43D is a cross-sectional view of the porous metal-backed glenoid implant of FIGS. 43A-43B.

FIG. 43E is a detailed view of the region A noted in FIG. 43D.

FIG. 43F is an illustration of a view of the porous metal-backed glenoid implant of FIGS. 43A-3B looking straight on to the articulation surface 1130 of the implant.

FIG. 43G is an illustration showing how various modular non-peripheral fixation features can be used in combination with the metal-backed glenoid implant of FIGS. 43A-43B.

FIG. 43H is a cross-sectional view of an implant converted to reverse configuration. The tapered cylindrical feature would assemble with a glenosphere. Additionally, there can be screw holes that go through this cylindrical attachment.

FIG. 44A is an isometric view illustration of a glenoid implant employing a circular bone engagement rim according to another embodiment.

FIG. 44B is a side view of the glenoid implant of FIG. 44A.

FIG. 44C is a cross-sectional view of the glenoid implant of FIG. 44A.

FIG. 44D is an isometric view illustration of a glenoid implant employing a circular bone engagement rim according to another embodiment.

FIGS. 44E-44F are cross-sectional view illustrations of variations of the glenoid implant of FIG. 44A.

FIG. 45A is an isometric view illustration of a glenoid implant employing a circular bone engagement rim according to another embodiment.

FIG. 45B is a side view illustration of the glenoid implant of FIG. 45A.

FIG. 45C is an isometric view illustration of a glenoid implant employing a circular bone engagement rim according to another embodiment.

FIG. 45D is a side view illustration of the glenoid implant of FIG. 45C.

FIG. 45E is an illustration showing how a glenoid may be prepared with a recess for receiving the glenoid implants shown in FIGS. 44A and 45A.

FIG. 45F is an illustration showing the glenoid implant of FIG. 44A or 45A implanted in a glenoid.

FIGS. 45G-45H are illustrations showing examples of cutting instruments for preparing a glenoid for receiving the glenoid implants shown in FIGS. 44A and 45A.

FIG. 46A is an isometric view illustration of a glenoid implant employing a circular bone engagement rim according to another embodiment.

FIG. 46B is a cross-sectional view illustration of the glenoid implant of FIG. 46A.

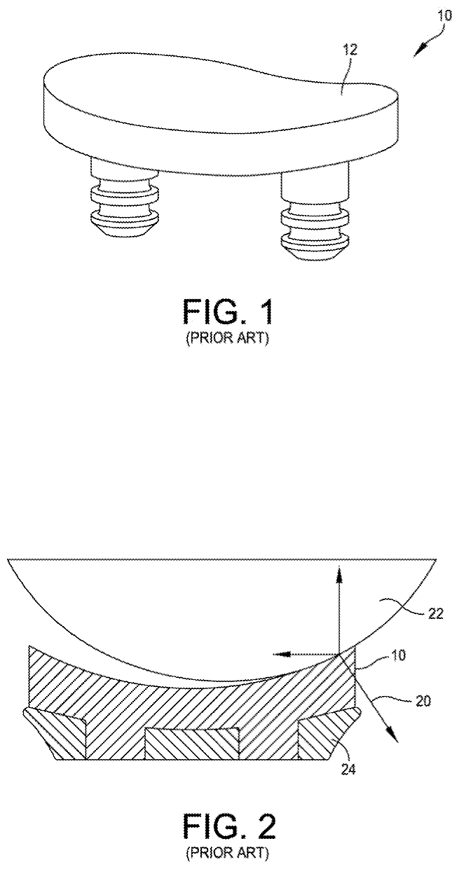

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

Provided herein are various improved glenoid implants that have articulation surface that is configured to engage with an anatomical humeral head or a humeral component of a shoulder replacement implant system. Therefore, references to “a humeral head” as used herein should be construed to include both an anatomical humeral head as well as implant humeral head.

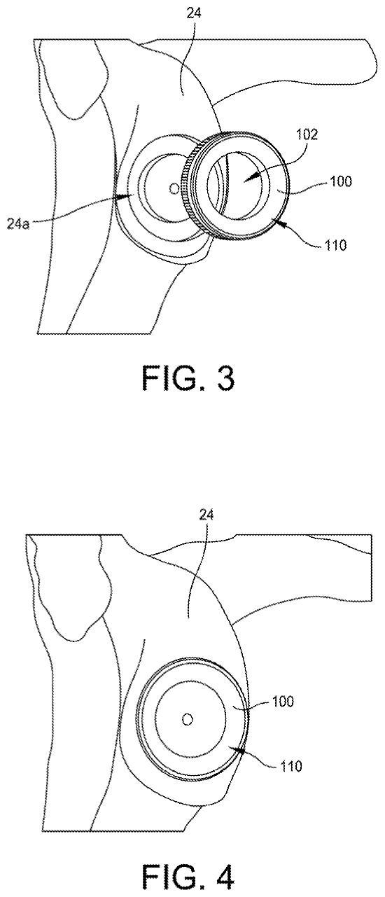

According to an embodiment illustrated in FIGS. 3-7, an inlay glenoid implant 100 is provided that has a ring-like structure. The ring-shaped glenoid implant 100 has a ring-shaped body 101 that comprises a hole 102. The ring-shaped body 101 comprises an articulation surface 110 provided on one side and a base surface 101a provided on the opposite side. The hole 102 extends completely through the implant 100 from the articulation surface 110 to the base surface 101a. When implanted into a glenoid 24, the articulation surface 110 faces outward from the glenoid 24 and replaces the natural articulation surface of the glenoid cavity. The articulation surface 110 is configured to engage with a humeral head.

The ring shape of this glenoid implant allows stabilization of the shoulder while minimizing bone reaming/removal. Minimizing bone reaming/removal can be beneficial for healing of the surgical site. The ring shape having a hole in the center allows the glenoid to support differing mating component curvatures while maintaining a continuous ring of contact. This differs from a conventional articulation surface that has a theoretical single point contact (not accounting for material deformation). Additionally, this nature of the ring contact offers a self-centering force to the mating humeral spherical head. Self-centering is good to help keep the humeral head from moving off the glenoid.

The hole 102 can be any desired shape and any size. For example, in some embodiments, the hole 102 can have a circular shaped outline as shown. In some other embodiments, the hole can be configured to have a polygon shaped outline. The polygon can be a regular or an irregular polygon. In some embodiments, the hole 102 can be configured as a patient specific irregular shaped hole that is customized to match the shape of an area of the patient's glenoid that is desirable to keep intact so that the ring-shaped implant 100 surrounds the area being saved.

The curvature of the articulation surface 110 can be configured to have any desired contour. In some embodiments, the curvature of the articulation surface 110 can be spherical.

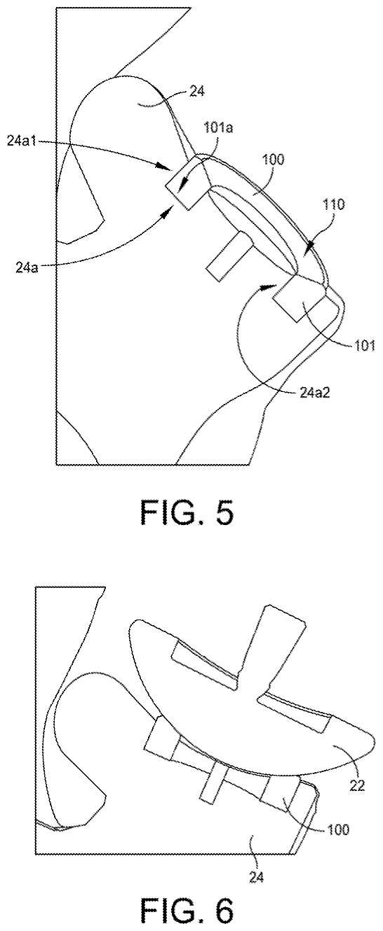

Referring to FIG. 3, the glenoid 24 is prepared with a ring-shaped recess 24a in which the ring-shaped glenoid implant 100 gets positioned. FIG. 4 shows the ring-shaped glenoid implant 100 that is seated in the ring-shaped recess 24a. FIG. 5 shows a cross-sectional view of the seated ring-shaped glenoid implant 100. The bottom surface 101a of the ring-shaped body 101 is in contact with the bottom surface of the ring-shaped recess 24a. The cross-section view in FIG. 6 shows a humeral head 22 engaging the articulation surface 110 of the ring-shaped glenoid implant 100.

The corresponding surfaces of the ring-shaped body 101 and the ring-shaped recess 24a are configured to provide intimate contact and enable the glenoid implant 100 to be securely seated in the recess 24a. The base surface 101a of the ring-shaped body 101 and the bottom surface of the ring-shaped trough 24a are contoured to match each other's contour to ensure that the two surfaces are intimately in contact when the glenoid implant 100 is inserted into the ring-shaped recess 24a. In the illustrated example, the base surface 101a and the bottom of the ring-shaped recess 24a are flat. In some embodiments, however, the base surface 101a can be concave, convex, or flat and the bottom surface of the ring-shaped trough 24a has a complementary contour. Similarly, the inner surface 105 and the outer surface 103 of the ring-shaped glenoid implant 100 are substantially orthogonal to the base surface 101a. The ring-shaped recess 24a comprises an inner surface 24a2 and an outer surface 24a1 that are substantially orthogonal to the bottom surface of the recess 24a. (See FIG. 5). When the glenoid implant 100 is inserted into the ring-shaped recess 24a for implantation, the corresponding surfaces of the glenoid implant 100 and the ring-shaped recess 24a come in contact with each other and securely hold the glenoid implant 100 in place.

In some embodiments, the ring-shaped glenoid implant 100 can be formed in whole or in part, particularly the portion that forms the articulation surface 110, of a synthetic material, such as, for example, polyethylene (e.g. ultra-high-molecular-weight polyethylene (UHMWPE)), polyether ether ketone (PEEK), etc. All references to UHMWPE herein includes all variants of UHMWPE in orthopedic application such as vitamin E diffused UHMWPE. In a preferred embodiment, the ring-shaped glenoid implant 100 can be formed in whole or in part, particularly the portion that forms the articulation surface 110, of hydrogel material.

The hydrogel material referred to herein refers to a three-dimensional solid resulting from cross-linked hydrophilic polymer chains formed of polyvinyl alcohol (PVA). The hydrogel material can comprise one or more other materials in addition to PVA, such as, for example, other hydrogels, other polymeric materials, additives, and/or the like. In some embodiments, the PVA content of the hydrogel in the implants disclosed herein can be about 40% by weight. The PVA content of the hydrogel can range from about 10% by weight to about 80% by weight, as appropriate for particular application.

The hydrogel can comprise water, saline, other liquids, combinations thereof, and/or the like. In some embodiments, saline may be preferred over water, because, under certain circumstances, saline can help maintain osmotic balance with surrounding anatomical tissues following implantation. The exact composition of the hydrogel component in an implant can be selected for optimal performance in a particular application to achieve the desired or required strength, load bearing capacity, compressibility, flexibility, longevity, durability, resilience, coefficient of friction, and/or other properties and characteristics.

In some embodiments, such hydrogel portion(s) of the ring-shaped glenoid implant 100 and all other embodiments of glenoid implants disclosed herein can be formulated for drug delivery and/or is seeded with growth factors and/or cells. In such embodiments, the hydrogel component can comprise one or more of the following: chondrocytes, growth factors, bone morphogenetic proteins, collagen, hyaluronic acid, nucleic acids, and stem cells. Such factors and/or any other materials included in the implants can help facilitate and/or promote long-term fixation of the implants at the joint site.

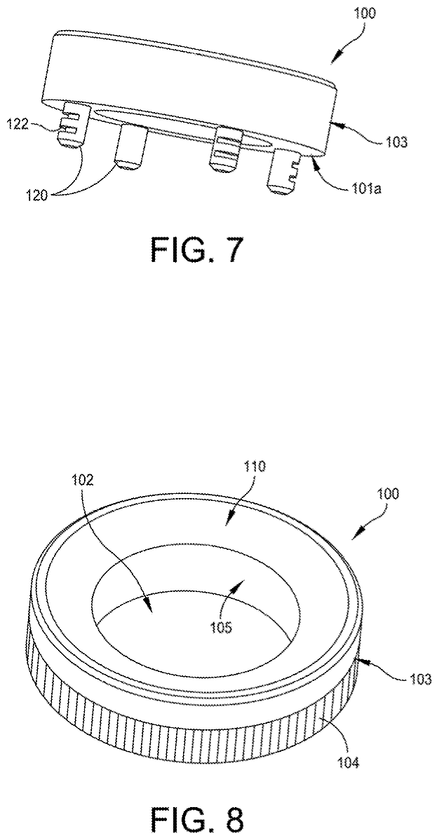

The ring-shaped glenoid implant can be affixed into the prepared glenoid 24 using a variety of methods. FIG. 7 shows an embodiment of the ring-shaped glenoid implant 100 can comprise one or more pegs 120 along the base surface 101a for securing the implant into the glenoid 24. Each of the pegs 120 can comprise one or more slots 122 for accommodating bone cement to secure the implant 100 in the bone 24. To use this embodiment of the ring-shaped glenoid implant the glenoid 24 would need to be prepared with corresponding holes for the pegs 120. The procedure for preparing the bone will be described below.

In FIG. 8, another embodiment of the ring-shaped glenoid implant 100 is shown that is configured for cementless fixation into the glenoid 24. In this embodiment, the glenoid implant 100 comprises at least a portion of the outer surface 103 that is coated with a porous trabecular metallic material 104, such as ADVANCE® BIOFOAM™ of Wright Medial Technology, Inc., for bone ingrowth into the glenoid implant. The structure of the coated trabecular metallic material resembles that of trabecular bone. In some embodiments, at least some portion(s) of the interior surface 105 of the ring-shaped glenoid implant 100 can also be coated with a trabecular metallic material.

In some embodiments of the ring-shaped glenoid implant 100 that is configured for cementless fixation, the bulk of the ring-shaped glenoid implant 100 that includes the bone-engaging base surface 101a can be formed of the porous trabecular metallic material and the articulation surface portion can be made of a hydrogel that is bonded to the porous trabecular material.

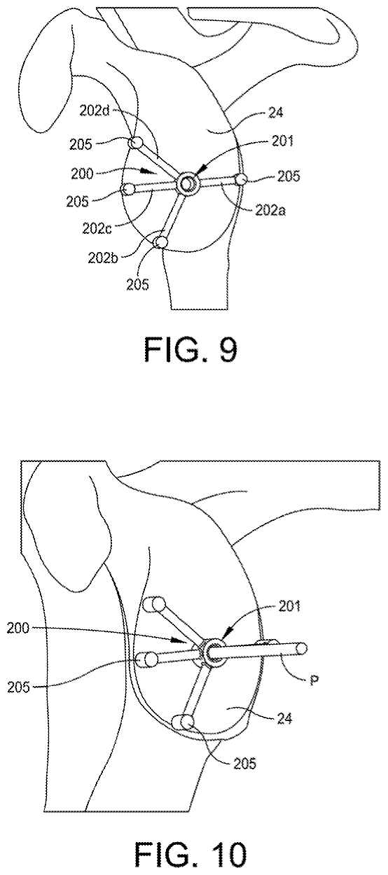

Referring to FIGS. 9 through 17, a corresponding procedure for preparing the glenoid 24 for receiving a ring-shaped glenoid implant 100 is disclosed. Shown in FIG. 9 is a drill guide 200 for identifying a center of the glenoid 24 that is positioned over the glenoid 24. The drill guide 200 comprises a guide hole 201 and a plurality of arms 202a, 202b, 202c, and 202d that radially extend from the guide hole 201 orthogonal to the central axis of the guide hole 201. Each of the arms 202a-202d are provided with an edge guide 205 at the arm's terminal end. As can be seen in the perspective view in FIG. 10, each of the edge guides 205 extend orthogonal to their respective arms and they are configured to be used to straddle the perimeter edge of the glenoid 24 as illustrated. As shown in FIG. 9, in the preferred embodiment of the drill guide 200, two of the arms 202a and 202c extend out from the guide hole 201 at 180° apart from each other. The remaining two arms 202b and 202d are positioned straddling the arm 202c. Because all of the arms extend out from the guide hole 201 orthogonal to the central axis of the guide hole 201, the arms are in the same plane.

The arrangement of the arms and their edge guides 205 allow the edge guides 205 to fit around the perimeter of the glenoid 24 as shown so that the guide hole 201 automatically locates the geometric center. To accommodate different size glenoid among the patients, the drill guide 200 can be provided in a variety of graduated sizes.

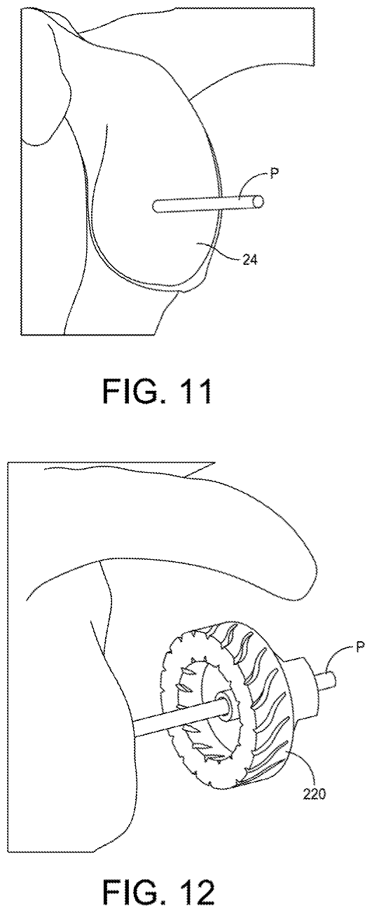

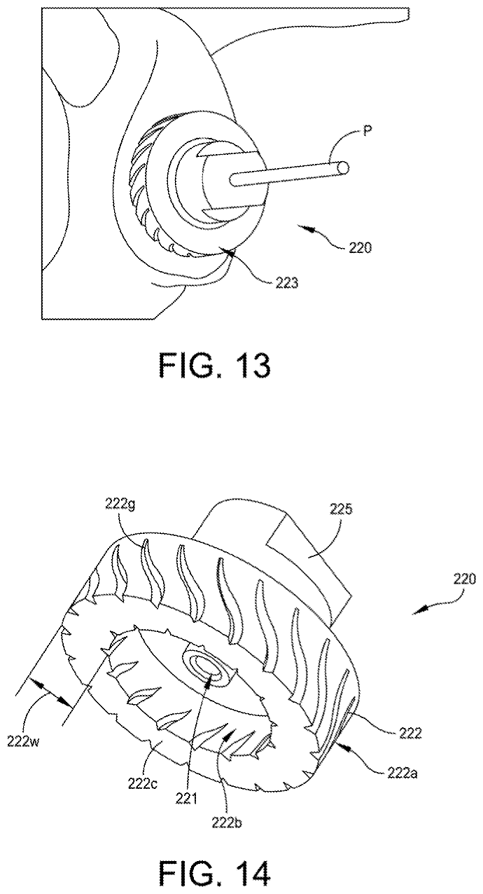

After the appropriately sized drill guide 200 is placed over the glenoid 24 as shown in FIG. 9, a hole is drilled into the glenoid 24 via the guide hole 201. Then, a pin P is inserted into the drilled hole as shown in FIG. 10. Referring to FIGS. 11 and 13, once the pin P is in place, the drill guide 200 is removed and a reamer 220 is used to carve the ring-shaped trough 24a into the glenoid 24.

The placement of the drill guide 200 on the glenoid 24 can be accomplished visually or with the aid of Wright Medical Technology's Blueprint™ 3D surgical planning system. Furthermore, the drill guide 200 can be a patient specific instrument fabricated using the Blueprint™ system.

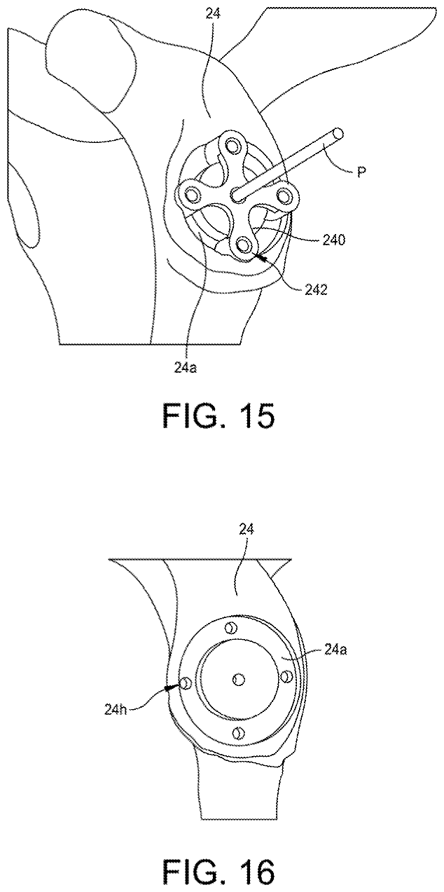

As shown in FIGS. 13 and 14, the reamer 220 is generally shaped like a bell saw and comprises a mandrel portion 223 and a cylindrical blade portion 222. The cylindrical blade portion 222 has a cutting end 222c that has a width 222w that creates trough 24a. The cylindrical blade portion 222 has an outer wall 222a and an inner wall 222b. The two circular edges defined by the cutting end 222c and the outer and inner walls 222a, 222b form the cutting edges. The outer and inner walls 222a, 222b can further comprise grooves 222g that aids in the cutting action as well as expulsion of the bone cutting debris during the reaming procedure. The shape and dimensions of the grooves 222g can be varied to optimize the reaming efficiency of the reamer 220. Provided at the center of the mandrel portion 223 is a hole 221 so that the reamer 220 can be placed over the pin P. The mandrel portion 223 comprises a driving tool engaging portion 225. The driving tool engaging portion 225 is configured to engage a driving tool, such as a surgical hand drill, that can turn the reamer 220 for the reaming action.

Where the glenoid implant 100 is the embodiment shown in FIG. 7 that has a plurality of pegs 120 for securing the implant into the glenoid 24, a drill guide 240 shown in FIG. 15 can be used to prepare the blind holes for receiving the pegs 120 in the bottom of the trough 24a. The drill guide 240 is configured to be slipped over the pin P so that the drill guide 240 is in alignment with the trough 24a. FIG. 16 is an illustration of a glenoid 24 that has been prepared with a trough 24a and blind holes 24h for receiving the glenoid implant 100 embodiment shown in FIG. 7.

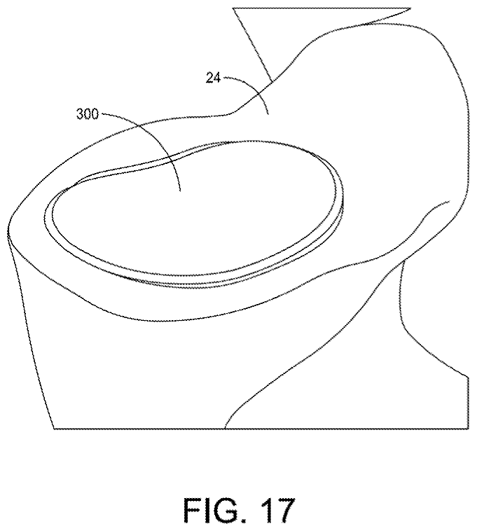

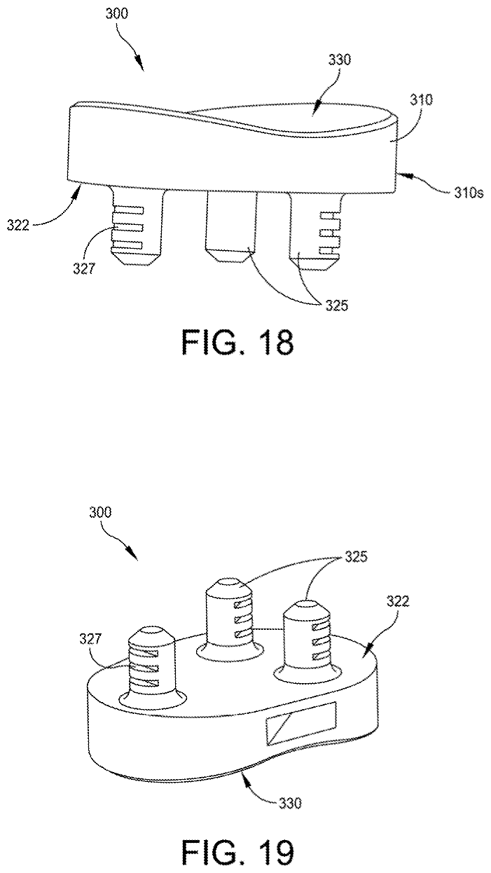

Referring to FIGS. 17-21, an embodiment of a glenoid implant 300 that is configured to be implanted into a glenoid in an inlay configuration is disclosed. FIG. 17 shows the glenoid implant 300 in an implanted state in a glenoid 24. FIG. 18 is a side view of an embodiment of the glenoid implant 300. FIG. 19 is an isometric view of the glenoid implant 300. The glenoid implant 300 comprises a body 310 comprising an articulation surface 330 and an anchor surface 322 on the opposite side. The glenoid implant 300 can be configured to have a shape that maximizes the articulation surface of the glenoid 24 that is replaced by the glenoid implant 300. The articulation surface 330 is contoured to replicate the anatomical articulation surface of the glenoid 24. In some embodiments, the anchor surface 322 comprises one or more fixation features such as posts, finned anchors, or keel, etc. extending therefrom. In the illustrate example shown, three anchors 325 extend from the anchor surface 322.

As shown in FIG. 17, the glenoid implant 300 is sized and shaped to be implanted into a glenoid 24 that is prepared with a recess to receive the glenoid implant 300. Inlay glenoid implant such as the glenoid implant 300 and the ring-shaped glenoid implant 100 are smaller than the full surface of the glenoid 24. Therefore, inlay configuration allows replacing just the defective or damaged portion of the glenoid's articulation surface and minimize disturbing the native glenoid bone material.

In some embodiments, each of the one or more anchors 325 can comprise one or more bone cement pockets 327. When the glenoid implant 300 is being secured into the corresponding recess prepared in the glenoid 24, bone cement can be used to enhance the securement of the implant. Each of the pockets 327 in the anchors 325 holds an amount of bone cement and assist in the securement of the glenoid implant 300.

In some embodiments, the glenoid implant 300 can be formed in whole or in part, particularly the portion that forms the articulation surface 110, of synthetic material, such as, for example, polyethylene (e.g. ultra-high-molecular-weight polyethylene (UHMWPE)), polyether ether ketone (PEEK), etc. In a preferred embodiment, the articulation surface 330 can be formed of the hydrogel material mentioned above.

In some embodiments, the glenoid implant 300 can be formed of a suitable surgical grade metal or metal alloy. Some examples are cobalt-chrome alloys and titanium alloys. When the glenoid implant 300 is made of a metal or metal alloy, the body 310 of the implant can be thinner because of the added stiffness and strength provided by metal. In the metal embodiments of the glenoid implant 300, the anchor surface 322 can be coated with porous trabecular metal coating mentioned above to promote ingrowth of bone tissue after the implant 300 is implanted. In some embodiments, portions of sidewall 310s (See FIG. 18) of the implant 300 can also be coated with porous trabecular metal coating.

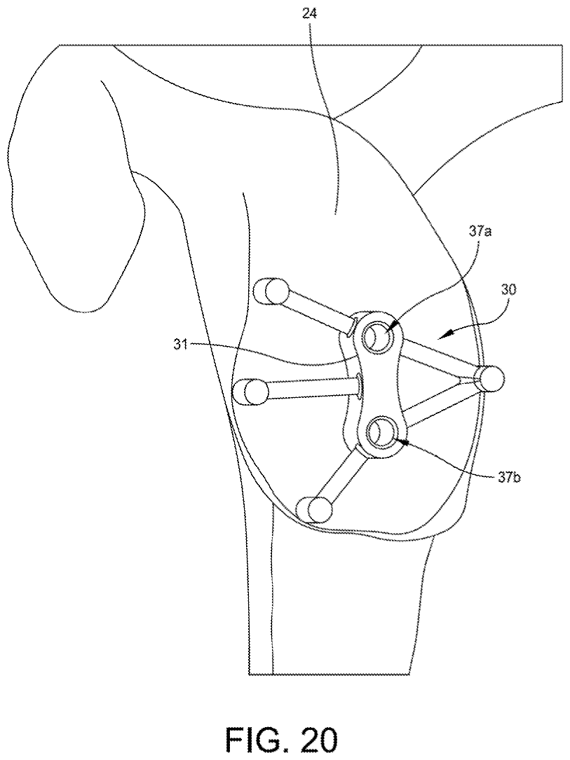

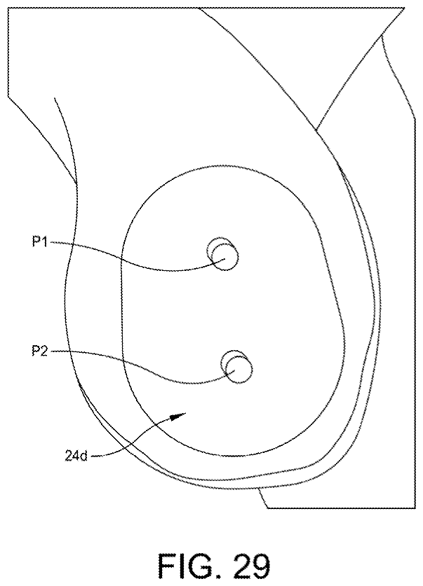

Referring to FIGS. 20-31, a corresponding procedure for preparing the glenoid 24 for receiving a glenoid implant 300 is disclosed. First, a drill guide 30 is placed on the glenoid 24 to locate the locations for drilling two blind holes for guide pins. The placement of the drill guide 30 on the glenoid 24 can be accomplished visually or with the aid of Wright Medical Technology's Blueprint™ 3D surgical planning system. In some embodiments, the drill guide 30 can be a patient specific instrument fabricated using the Blueprint™ 3D surgical planning system.

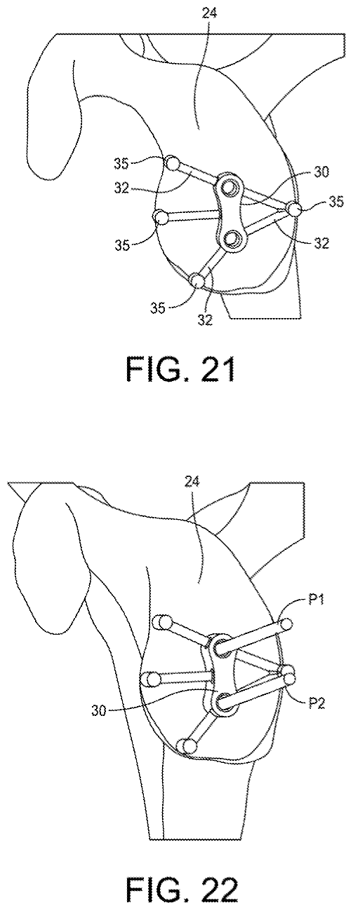

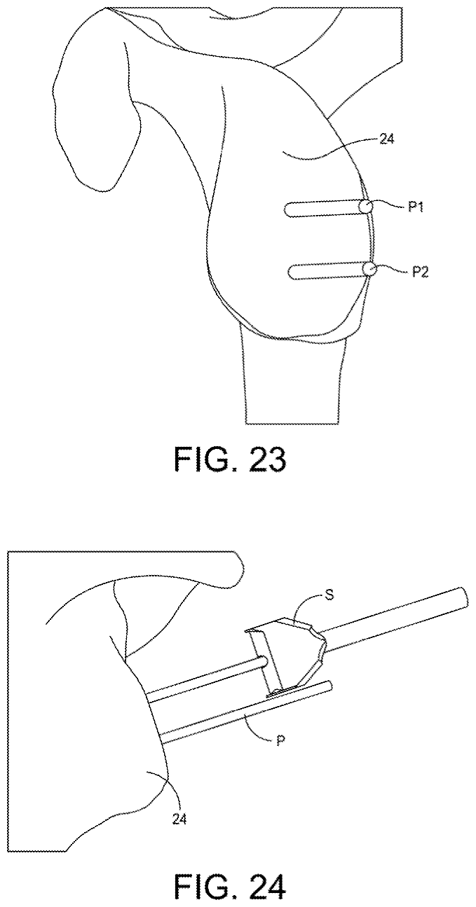

The drill guide 30 comprises a main body 31 that is provided with two drill guide holes 37a, 37b. The main body 31 comprises a plurality of arms 32 that extend outward from the main body 31 that terminate with edge guides 35 provided at the ends of the arms 32. Each of the edge guides 35 extend orthogonal to their respective arms and they are configured to be used to straddle the perimeter edge of the glenoid 24 as illustrated in FIGS. 20 and 21. The drill guide 30 can be provided in a variety of sizes to accommodate different size glenoid in different patients. Positioning an appropriately sized drill guide 30 on a glenoid 24 will position the two drill guide holes 37 in the desired position. Once the drill guide 30 is in the desired position, two blind holes are drilled into the glenoid 24 using the guide holes 37. Next, two guide pins P1, P2 are placed into the drilled blind holes in the glenoid 24 and the drill guide 30 is removed. See FIGS. 22-23.

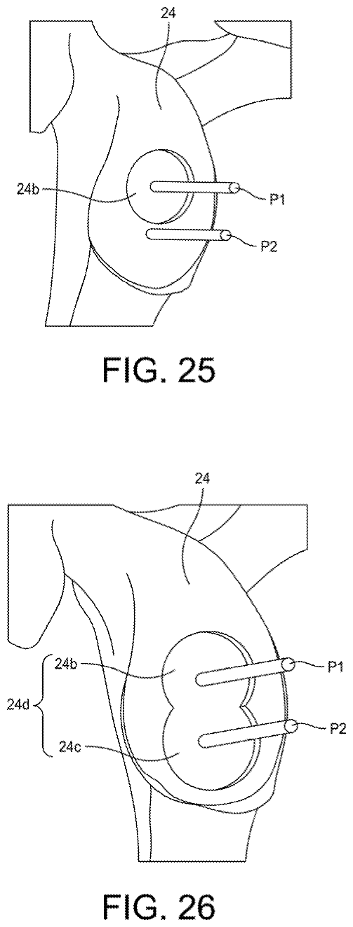

Next, a series of bone reaming procedure is carried out to form an appropriately shaped recess in the glenoid 24 to receive the glenoid implant 300. Referring to FIG. 24, a spade drill S, guided by the pins P1, P2, is used to form two, wide and shallow, blind holes (e.g. a recess). FIG. 25 shows first shallow, blind hole 24b thus formed. In the illustrated example, the guide pin P1 was used first to form the first blind hole 24b but the other guide pin P2 could have been used first. This procedure is repeated over the guide pin P2 to form a second shallow blind hole 24c as shown in FIG. 26. The two shallow blind holes 24b and 24c overlap as shown forming a single shallow recess 24d. The depth of the shallow blind holes are determined to accommodate the thickness of the glenoid implant 300.

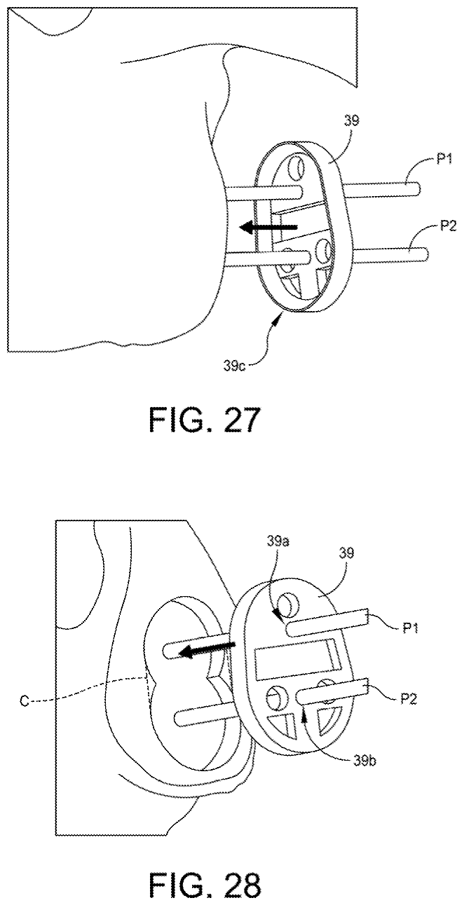

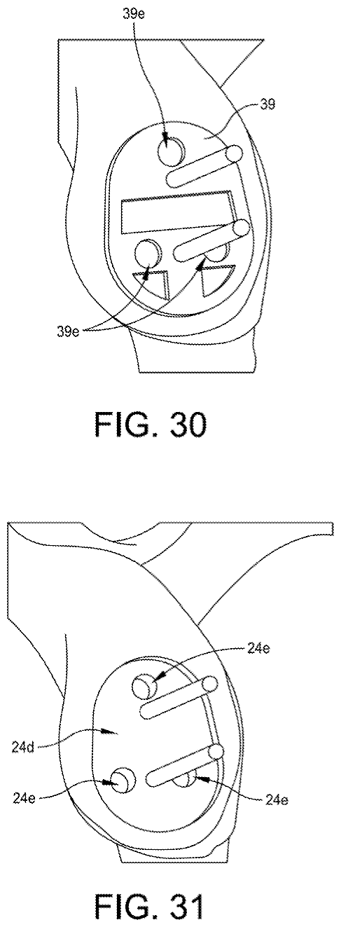

Referring to FIGS. 27-29, a cookie cutter type bone cutting device 39 is applied to create the final outline for the shallow recess 24d to receive the glenoid implant 300. The bone cutting device 39 has a cutting edge 39c that is shaped to cut the bone between the two shallow blind holes 24b, 24c along the dotted line C shown in FIG. 28. This removes the excess bone material between the two shallow blind holes 24b, 24c and form a recess 24d in the glenoid 24 having an outline of the glenoid implant 300 as shown in FIG. 30. To help guide and align the cookie cutter type bone cutting device 39, the bone cutting device can be provided with two guide holes 39a and 39b that are appropriately sized and located on the bone cutting device 39 to be slid over the pins P1, P2. Once the bone cutting device 39 is in place with the cutting edge 39c contacting the intended cutting line C, the bone cutting device 39 can be tapped down to cut into the bone.

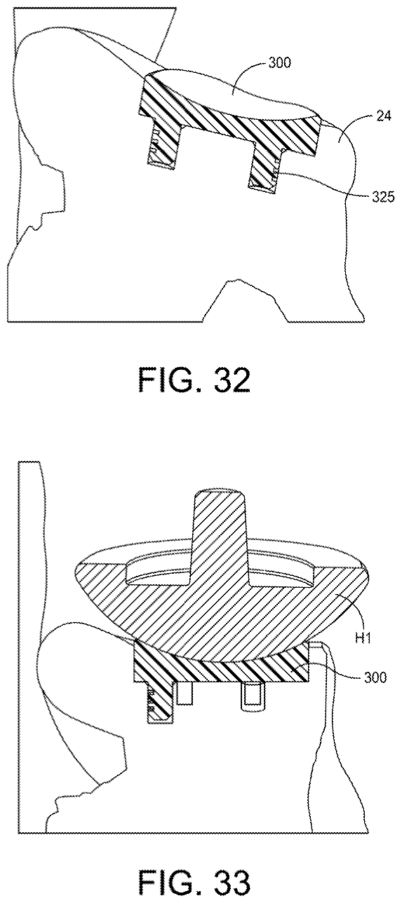

Referring to FIG. 30, the bone cutting device 39 can further comprise additional drill guide holes 39e that can be used to drill blind holes for receiving the anchors 325 that can be provided on the glenoid implant 300. FIG. 32 is a sectional view of the implanted glenoid implant 300. FIG. 33 shows an example of a humeral head implant H1 interacting with the articulation surface of the glenoid implant 300.

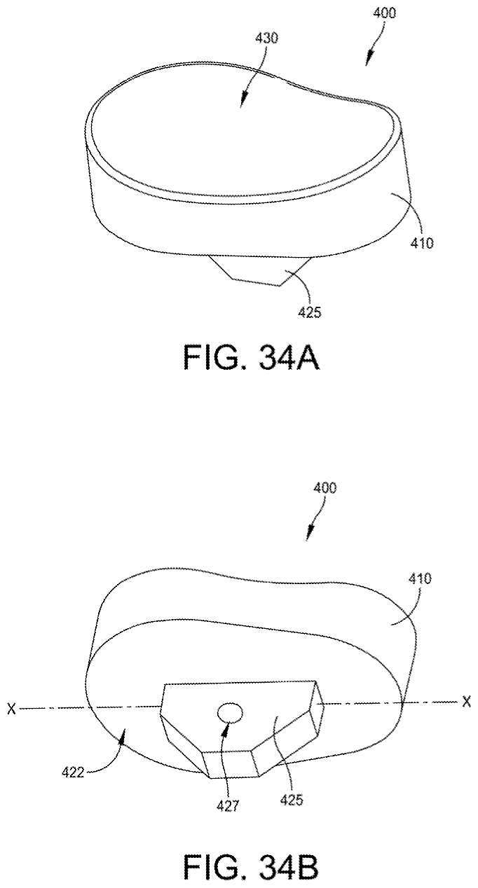

Referring to FIGS. 34A-34B, another embodiment of a glenoid implant 400 that is configured to be implanted into a glenoid in an inlay configuration is disclosed. Similar to the glenoid implant 300, the implant 400 can be configured with a shape that maximizes the articulation surface area of the glenoid 24 that is replaced by the implant 400. FIG. 34A shows an isometric view of the top side, i.e. an articulation surface 430 side, of the glenoid implant 400. FIG. 34B is an isometric view of the glenoid implant 400 from the bottom side. The glenoid implant 400 comprises a body 410 having the articulation surface 430 and an anchor surface 422 on the opposite side. The articulation surface 430 s contoured to emulate the natural articulation surface of the glenoid 24. The articulation surface 430 includes a concave profile that is intended to cooperate in the anatomical manner with the humeral head whether it be a natural one or a prosthetic one. The anchoring surface 422 can be coated with a porous trabecular metallic material such as Wright Medical Technology's ADAPTIS™ that can promote bone tissue ingrowth to enhance bonding of the glenoid implant 400 to glenoid after the implant is implanted to a prepared glenoid.

Extending from the anchor surface 422 is an anchoring keel 425. The anchoring keel 425 is configured to securely anchor the implant 400 in the glenoid 24 in an inlay configuration and to minimize or prevent rocking of the implant 400 after it is implanted. The keel 425 extends along longitudinal axis X-X. The keel 425 preferably includes a longitudinal dimension or length that is greater than its transverse dimension or width. The keel 425 can have a structure of an elongated ridge or upstanding structure attached to the glenoid implant's body 410 with a length oriented along a longitudinal axis of the glenoid implant 400. The keel 425 can have a length along the longitudinal axis X-X that is greater than, less than, or the same as the body 410 of the glenoid implant 400. The keel 425 can have generally planar sides, which can include a variety of protrusions, recesses, anchor members, and holes.

In the illustrated example shown in FIG. 34B, the keel 425 includes a transverse hole 427, allowing the creation of a cement bridge for fixing the implant 400, if it is cemented. If the glenoid implant 400 is not cemented, the hole 427 is likely to allow the creation of a bone bridge. In some embodiments, the hole 427 may be used to receive one or more fasteners, such as bone screws.

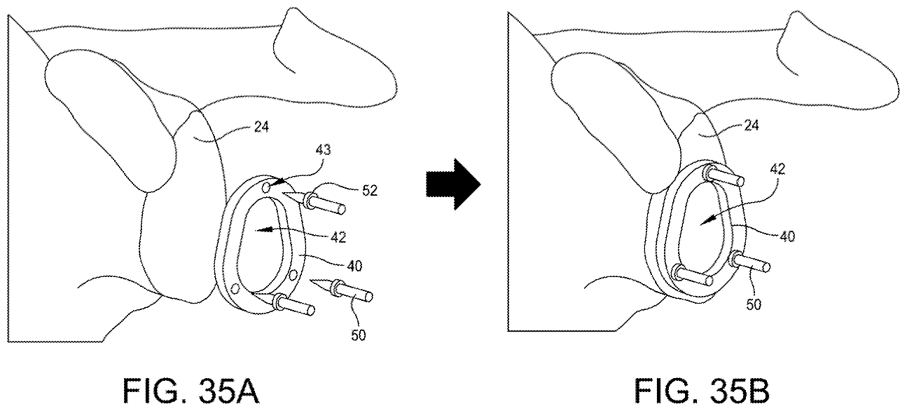

The process of preparing the glenoid 24 to receive the glenoid implant 400 will now be described in conjunction with FIGS. 35A-36B. The process involves using a milling guide 40 to remove a portion of the glenoid 24 from the articulation surface of the glenoid 24 to form a shallow recess that can receive the implant 400. The milling guide 40 has a ring-like structure with an opening 42 in the center. Referring to FIG. 36A, after the milling guide 40 is placed on the glenoid 24 at a desired location, the opening 42 allows access to the area of the glenoid 24 for removal using a reamer bit 55. As shown in the caption for FIG. 36A, an arbitrary example pathway for the milling operation using a reamer bit can be as shown by the arrows indicated inside the opening 42. The direction of the movement for the reamer bit can be in any direction as long as the reamer is maintained within the opening 42. If some bone material remains at the site after using the reamer bit, especially in the center of the opening 42, the bone material can be removed with a rongeur drill bit to finish the preparation of the bone.

Referring to FIG. 36B, the milling operation results in a shallow recess 24f formed in the glenoid 24 that is shaped to receive the glenoid implant 400. The slot 24g for the keel can be formed using a punch tool.

The shape of the opening 42 can be any desired shape. It can be circular, oval, piriform, etc. The back side of the milling guide 40 that comes in contact with the glenoid can be flat that can be used universally on all patients or the surface can be pre-formed with a customized surface that has a contour that is patient specific. The milling guide 40 also comprises a plurality of holes 43 provided on its ring-like structure for accommodating fixation pins/tacks 50 to temporarily affix the milling guide 40 in place during the subsequent reaming procedure. In the illustrated example, the milling guide 40 has three such holes 43. The pins 50 comprise a shoulder 52 in the middle of its length that has a diameter larger than the holes 43 so that after the pins 50 are in place as shown in FIG. 35B, the milling guide 40 cannot slide up the pins 50.

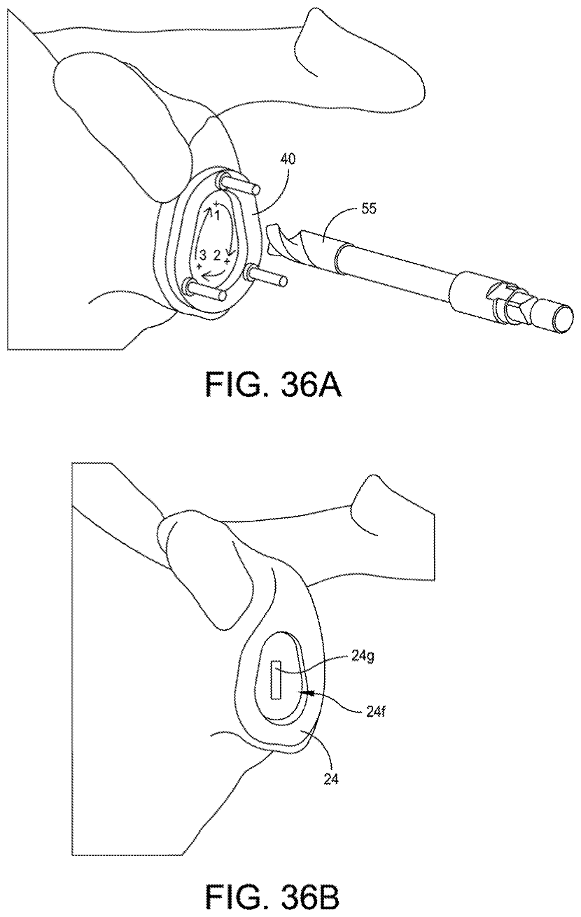

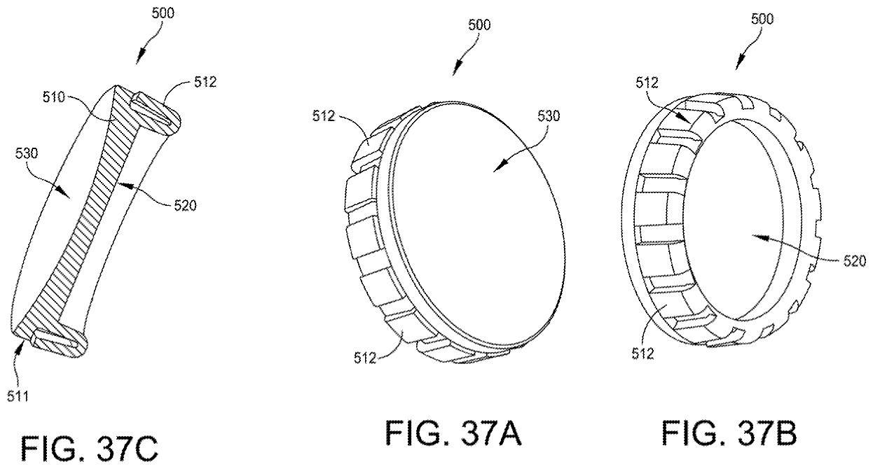

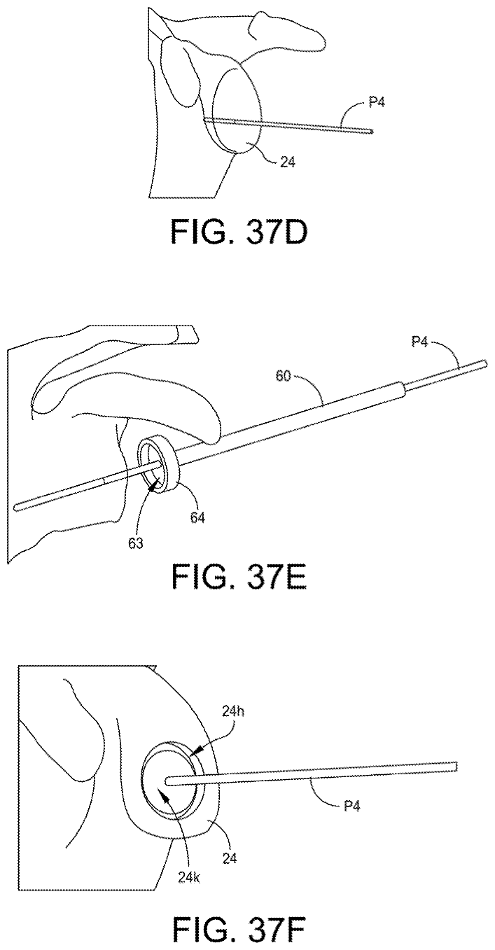

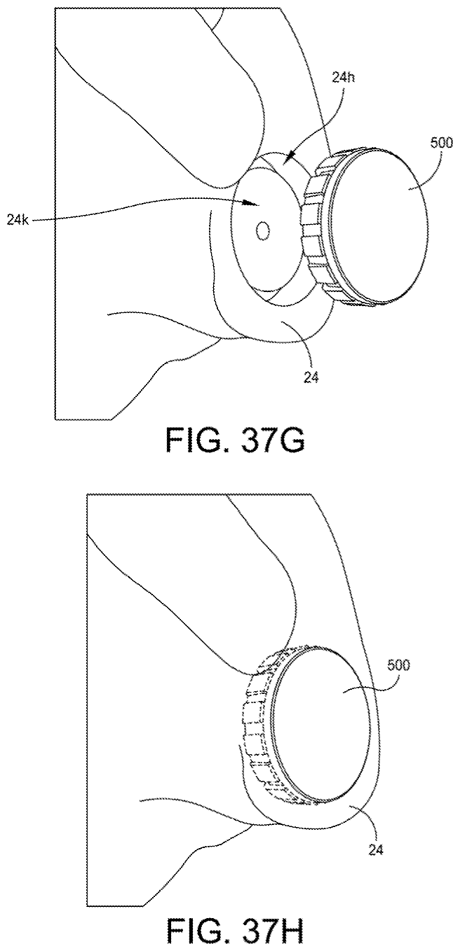

Referring to FIGS. 37A-37C, a glenoid implant 500 according to another embodiment is disclosed. The glenoid implant 500 is configured to be securely press fitted into a recess prepared into a glenoid to replace damaged natural articulation surface. The glenoid implant 500 comprises a circular body 510 having an articulation surface 530 on one side, an anchoring surface 520 on the opposite side, and an annular side wall 511 extending around the periphery of the circular body 510. The articulation surface 530 is contoured with a generally concave surface that emulates the natural articulation surface of a glenoid. The articulation surface 530 is configured to cooperate in the anatomical manner with a humeral head.

The articulation surface 530 has a surface finish that is appropriate for an articulation surface that is intended to engage a humeral head. The anchoring surface 520 is generally flat and is intended to contact the cortical bone of a prepared glenoid. The anchoring surface 520 can be coated with a porous tarbecular metallic material such as Wright Medical Technology's ADAPTIS™ that can promote bone tissue ingrowth to enhance bonding of the glenoid implant 500 to glenoid after the implant is press fitted into a recess prepared in a glenoid.

To enable the press fitting feature of the glenoid implant 500, the annular side wall 511 comprises a plurality of retaining legs 512 that are provided along the annular side wall 511. glenoid implant 500. In some embodiments, the retaining legs 512 are extensions of the annular side wall 511 that are folded over towards the articulation surface 530 so that each of the retaining legs 512 are formed as a U-shaped leaf spring as shown in the cross-sectional view in FIG. 37C. Each of the U-shaped leaf spring form of the retaining legs 512 is in an open configuration and is elastically compressible in the radially inward direction. The U-shape is open toward the articulation surface 530 side of the implant 500. To press fit the implant 500 into a shallow recess 24h (See FIG. 37G) formed in a glenoid, the recess should have a diameter that is slightly smaller than the outer diameter of the glenoid implant 500 defined by the outer surface of the retaining legs 512. Thus, when the glenoid implant 500 is inserted into the shallow annular recess formed in a glenoid, the retaining legs 512 are elastically compressed by the side walls of the circular recess as the implant 500 is squeezed in. The outward spring force exerted against the side walls of the annular recess 24h by the retaining legs 512 securely holds the implant 500 in the recess. The retaining legs 512 are compressed into the bone improving the quality of the anchoring. Because of the U-shaped configuration of the retaining legs 512 that is open toward the surface of the glenoid 24, the more the implant 500 is pulled outward from the recess, the more the retaining legs 512 will expand radially outward into the surrounding bone and securely hold the implant 500 in the implanted position.

The glenoid implant 500 can be made of a metal such as titanium, CoCr alloy, or a high modulus polymer such as UHMW polyethylene. Preferably, the glenoid implant 500 is integrally formed as a single piece construction. Although not necessary, preferably, the retaining legs 512 and the circular body 510 are formed from a single material. The thickness and diameter of the glenoid implant 500 can be selected to be any desired value for the condition of the surgical site.

In the illustrated example shown in FIG. 37A-37C, the glenoid implant 500 has a generally circular or disk-like outline shape. The glenoid implant 500, however, is not limited to such circular shape and can be provided to have any desired non-circular outline shape. For example, the glenoid implant 500 can have a glenoid-like outline shape similar to the implant 300 shown in FIG. 18 and implant 400 shown in FIG. 34A.

FIGS. 37D-37F are illustrations showing an example of a procedure for preparing a glenoid to receive the glenoid implant of FIGS. 37A-37B. Referring to FIG. 37D, a pin P4 is placed at a desired location in a glenoid 24. An appropriate pin guide instrument or Wright Medical Technology's Blueprint™ 3D surgical planning system can be used to place the pin P4 at the desired location. Referring to FIG. 37E, Next, a cannulated 2-in-1 reamer 60 is slid over the pin P4 to form a circular recess in the glenoid 24 that includes a circular substantially flat surface 24k surrounded by an annular recess 24h along the perimeter as shown in detail in FIG. 37F. The circular flat surface 24k is formed to accommodate the anchoring surface 520 of the implant 500 when the implant is press-fitted into the recess. The annular recess 24h receives the retaining legs 512 of the implant 500. The reamer 60 comprises an annular cutting ring 64 with a disc shaped body 63 having a circular flat surface on the bone-facing side. The circular flat surface on the bone-facing side of the cutting ring 64 is also an abrasive surface that cuts the glenoid. Referring to FIG. 37G, after the circular recess is formed, the pin P4 is removed and the glenoid implant 500 is press-fit into the recess. FIG. 37H shows the fully seated implant 500. FIG. 37I is a cross-sectional view of the fully seated glenoid implant 500.

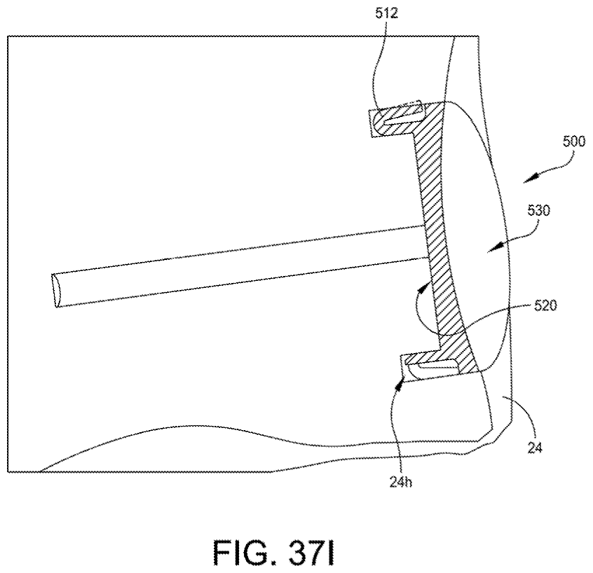

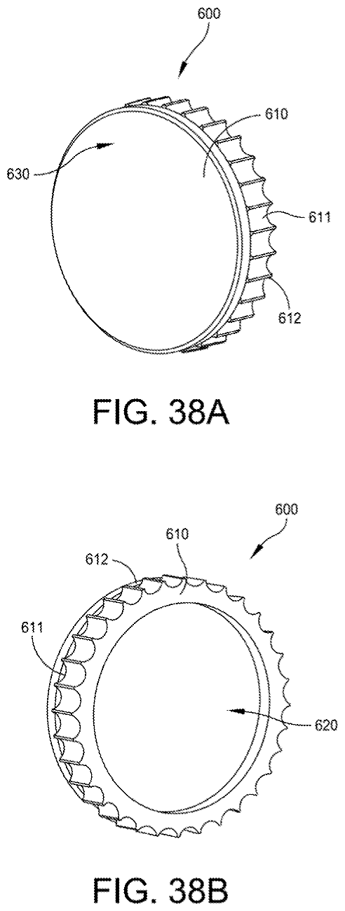

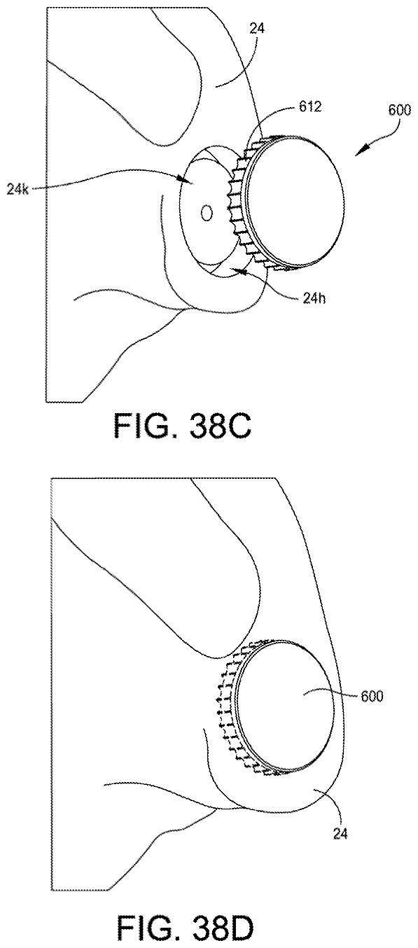

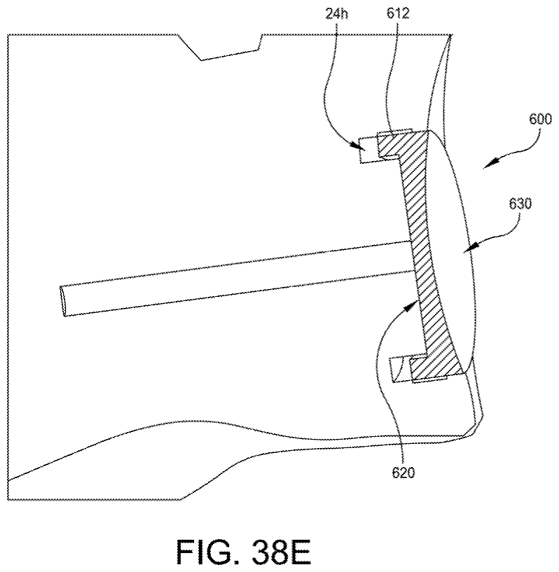

Referring to FIG. 38A-38B, a press-fit glenoid implant 600 according to another embodiment is provided. The glenoid implant 600 is configured to be securely press fitted into a recess prepared into a glenoid to replace damaged natural articulation surface. The glenoid implant 600 comprises a circular body 610 comprising an articulation surface 630 on one side, a base surface 620 on the opposite side, and an annular side wall 611 extending around the periphery of the circular body 610. The annular side wall 611 is a fluted surface that comprises a plurality of grooves with blades 612 formed in between two adjacent grooves. The glenoid implant 600 is intended to be press-fit into a circular recess prepared in the glenoid. 24. Similar to the circular recess shown in FIGS. 37F-37G for the glenoid implant 500, the circular recess prepared for the glenoid implant 600 also comprises a circular flat surface 24k surrounded by an annular recess 24h to accommodate the contour of the base surface 620 of the press-fit glenoid implant 600. This can be seen in the cross-sectional view in FIG. 38E of the press-fit glenoid implant 600 fully seated in the circular recess. The plurality of blades 612 interfere with the surrounding bone to accomplish a secure press-fitting.

The articulation surface 630 is contoured with a generally concave surface that emulates the natural articulation surface of a glenoid. The articulation surface 630 is configured to cooperate in the anatomical manner with the humeral head whether it be a natural one or a prosthetic one.

The articulation surface 630 has a surface finish that is appropriate for an articulation surface that is intended to engage a natural humeral head or a prosthetic humeral head. The anchoring surface 620 is generally flat and is intended to contact the cortical bone of a prepared glenoid. The anchoring surface 620 can be coated with a porous trabecular metallic material such as Wright Medical Technology's ADAPTIS™ that can promote bone tissue ingrowth to enhance bonding of the glenoid implant 600 to glenoid after the implant is press fitted into a recess prepared in a glenoid.

The procedure for preparing a glenoid 24 for implanting the press-fit implant 600 into the glenoid 24 is similar to the procedure illustrated in FIGS. 37D-F discussed above. The procedure is used to form a circular recess that comprises the circular flat surface 24k surrounded by the annular recess 24h. Then, the implant 600 is press-fit into the circular recess. FIG. 38D and the cross-section in FIG. 38E show illustrations of a fully seated glenoid implant 600.

The glenoid implant 600 can be made of a metal such as titanium, CoCr alloy or PEEK. Preferably, the glenoid implant 600 is integrally formed as a single piece construction. The thickness and diameter of the glenoid implant 600 can be selected to be any desired value for the condition of the surgical site.

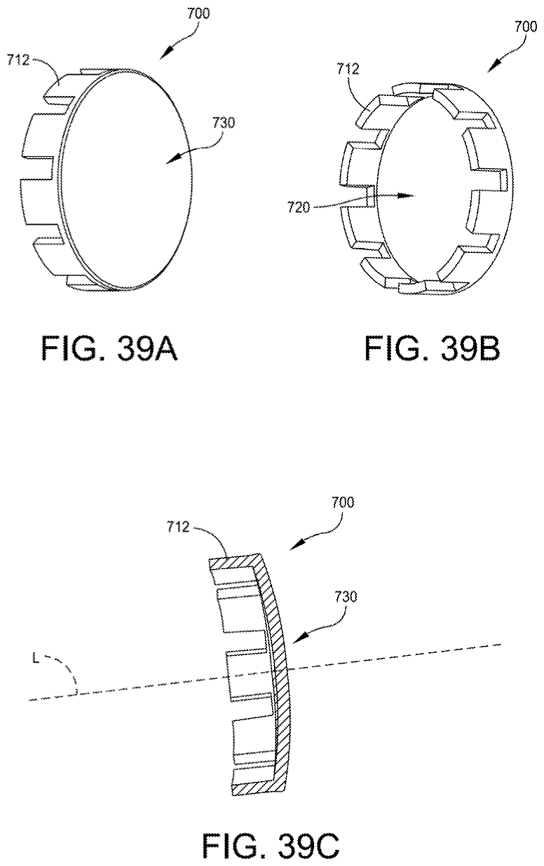

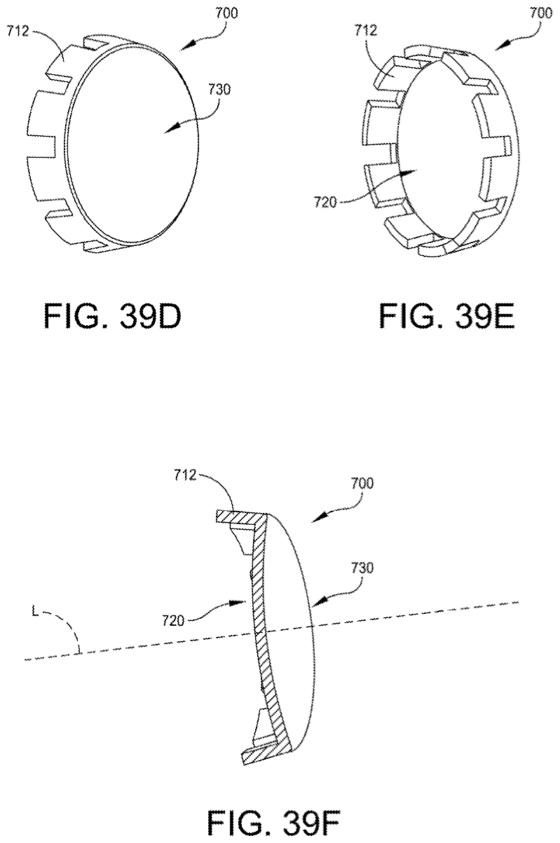

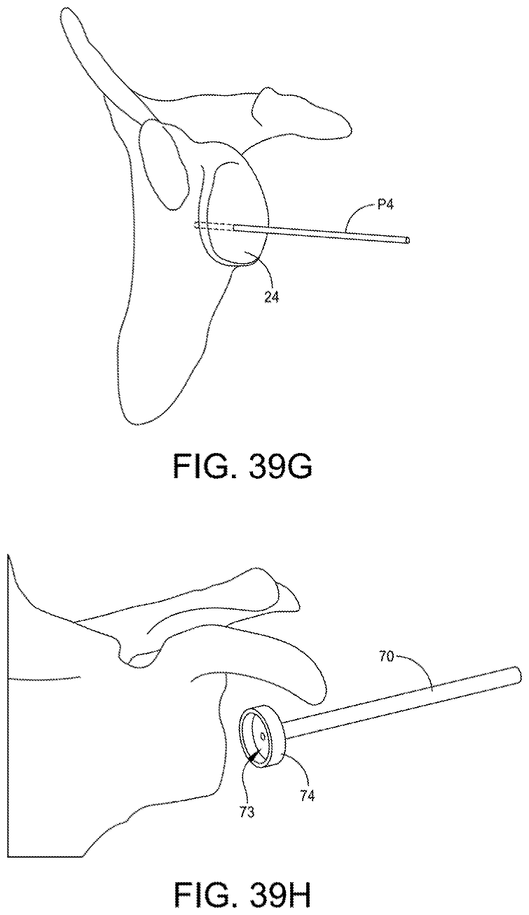

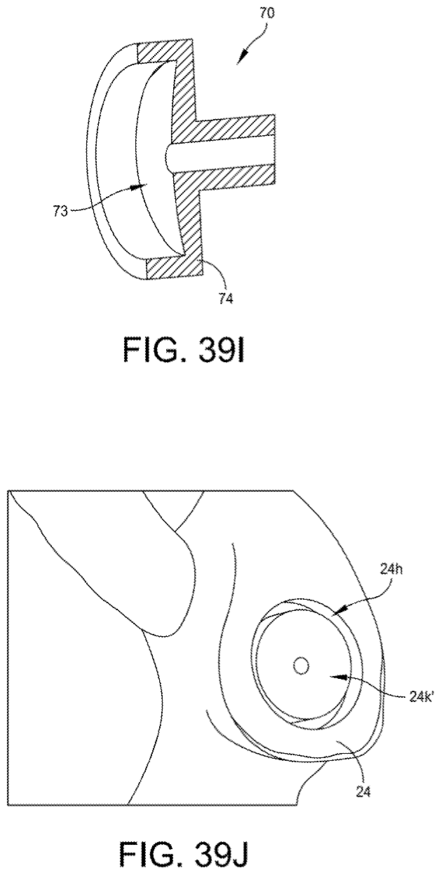

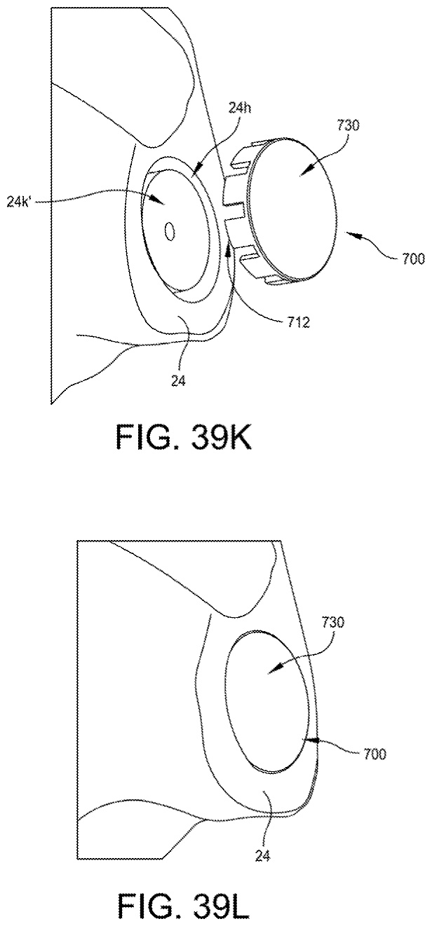

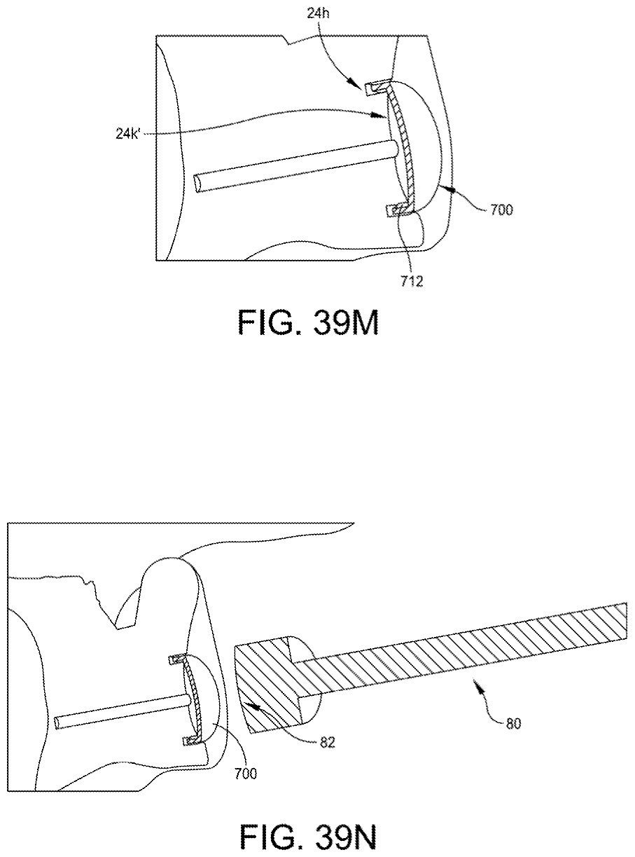

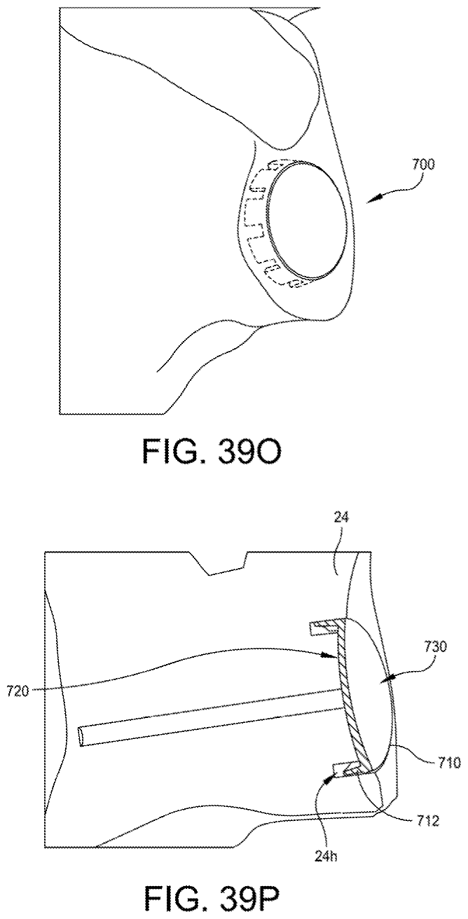

FIGS. 39A-39F are illustrations showing a glenoid implant 700 according to another embodiment of the present disclosure. The glenoid implant 700 comprises a circular body 710 comprising an articulation surface 730 on one side, an anchoring surface 720 on the opposite side, and a plurality of flexible legs 712 extending from the periphery of the circular body 710 toward the direction away from the articulation surface 730. Thus, the overall shape of the glenoid implant 700 generally resembles a bottle cap.

The glenoid implant 700 can be transformed from its pre-implant configuration to its implanted configuration by plastic deformation. FIGS. 39A-39C are illustrations showing the glenoid implant 700 in its pre-implant configuration and FIGS. 39D-39F are illustrations showing the glenoid implant 700 in its implanted configuration.

In the pre-implant configuration, the circular body 710 is in a shallow dome-like configuration so that the articulation surface 730 is convex and the anchoring surface 720 on the opposite side is concave as shown in FIGS. 39A-39C. Preferably, the flexible legs 712 extend from the periphery of the circular body 710 while being substantially parallel to or towards the longitudinal axis L of the implant 700. The longitudinal axis L is defined through the center of the circular body 710. Being substantially parallel here means at parallel or almost parallel. This configuration can be seen in the cross-sectional view in FIG. 39C. Maintaining the flexible legs 712 substantially parallel to the longitudinal axis L allows the implant 700 to be inserted into the glenoid 24 prepared with a recess.

In the implanted configuration, the circular body 710 of the implant has been plastically deformed so that it is now curved in opposite direction from its pre-implant configuration. Now the articulation surface 730 is concave and the anchoring surface 720 on the opposite side is now convex as shown in FIGS. 39D-39F. In this implanted configuration, because of the curved direction of the circular body 710, the flexible legs 712 around the periphery are now radially extending outward (i.e., away from the longitudinal axis L). This configuration helps secure the glenoid implant 700 inside the recess prepared in the glenoid 24 as will be discussed further below.

Referring to the illustrations of FIGS. 39G-39L, a procedure for preparing a glenoid 24 and implanting the glenoid implant 700 is now described. First, a recess is formed in the glenoid 24 to receive the glenoid implant 700 using a procedure illustrated in FIGS. 39G FIGS. 37D-37F. Referring to FIG. 39G, a pin P4 is placed at a desired location in the glenoid 24. An appropriate pin guide instrument or Wright Medical Technology's Blueprint™ 3D surgical planning system can be used to place the pin P4 at the desired location based on where the glenoid implant 700 should be centered. Referring to FIG. 39H, next, a cannulated 2-in-1 reamer is slid over the pin P4 to create a circular recess for receiving the glenoid implant 700. The reamer 70 comprises an annular cutting ring 74 with a disc shaped body having a circular abrading surface 73 on the bone-facing side. The circular abrading surface 73 is not flat but has a curvature as shown in the detailed cross-sectional view in FIG. 39I. This curved surface 73 is convex toward the bone surface it is grinding. Therefore, the 2-in-1 reamer 70 forms a recess in the glenoid 24 that includes a circular concave surface 24k surrounded by an annular recess 24h along the perimeter as shown in detail in FIG. 39J. The circular concave surface 24k is formed to accommodate the anchoring surface 720 of the implant 700 when it is in the implanted configuration.

Referring to FIGS. 39K and 39L, after the recess of appropriate size is formed in the glenoid 24, the glenoid implant 700 in the pre-implant configuration is inserted into the recess 24h. The flexible legs 712 of the implant 700 are received into the annular recess 24h. As shown, the articulation surface 730 is convex. Because the flexible legs 712 extend substantially parallel to or towards the longitudinal axis L of the implant 700, they do not interfere when inserted into the recess 24h. FIG. 39M shows a cross-sectional view of the glenoid implant 700 after it is inserted into the recess.

Referring to FIG. 39N, next, an impactor 80 is used to press down onto the articulation surface 730 to plastically deform the implant 700 into its implanted configuration. The impactor 80 comprises a convex tip 82 that has a curvature that substantially matches the convex curvature of the articulation surface 730 after the implant 700 is deformed into its implanted configuration. FIGS. 390 and 39P show the implant 700 after it has been deformed into its implanted configuration. The implant body 710 is now deformed into the implanted configuration so that the articulation surface 730 is concave. The anchoring surface 720 on the opposite side of the implant 700 is now convex and in contact with the concave surface of the circular concave surface 24k of the recess in the glenoid 24.

In the implanted configuration of the glenoid implant 700, the articulation surface 730 is contoured with a generally concave surface that emulates the natural articulation surface of a glenoid. The articulation surface 730 is configured to cooperate in the anatomical manner with the humeral head whether it be a natural one or a prosthetic one.

The articulation surface 730 has a surface finish that is appropriate for an articulation surface that is intended to engage a natural humeral head or a prosthetic humeral head. The anchoring surface 720 is generally flat and is intended to contact the cortical bone of a prepared glenoid. The anchoring surface 720 and/or the flexible legs 712 can be coated with a porous trabecular metallic material such as Wright Medical Technology's ADAPTIS™ that can promote bone tissue ingrowth to enhance bonding of the glenoid implant 700 to glenoid after the implant is press fitted implanted.

The implant 700 can be made of a metal such as CoCr, Nitinol, or titanium. Preferably, the glenoid implant 700 is integrally formed as a single piece construction. Although not necessary, preferably, the retaining legs 712 and the circular body 710 are formed from a single material. The thickness and diameter of the glenoid implant 700 can be selected to be any desired value for the condition of the surgical site. Because of the high modulus of the material forming the implant 700, when the implant is being deformed into the implanted configuration from the pre-implant configuration, the transition happens in a snap as the force exerted by the impactor 80 on the convex anchoring surface 730 reaches a threshold level and the implant's body 710 pops from the pre-implant configuration to the implanted configuration.

As mentioned previously, when the implant 700 is in its implanted configuration shown in FIGS. 39D-39F, the flexible legs 712 radially flare outward. Thus, as shown in the cross-sectional view in FIG. 39P, when the implant 700 is transformed into its implanted configuration while seated within the recess in the glenoid 24, the legs 712 are butted against the outer wall of the annular recess 24h as they flare outward. This secures the implant within the recess. The porous trabecular metallic material such as Wright Medical Technology's ADAPTIS™ that can be coated on the legs 712 and/or the anchoring surface 720 further enhance the long term securement of the implant 700 in the glenoid 24.

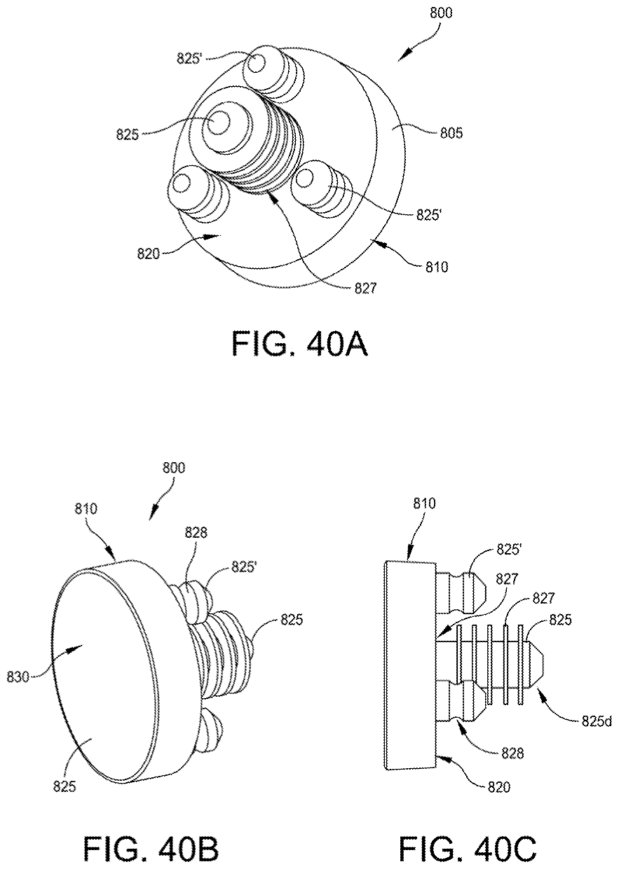

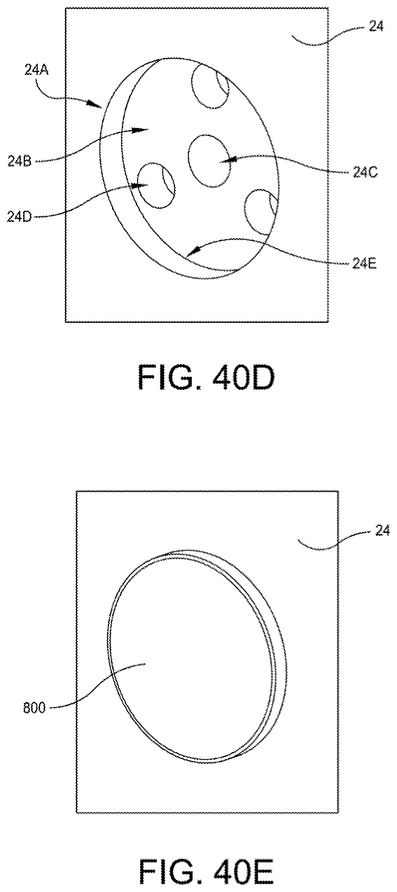

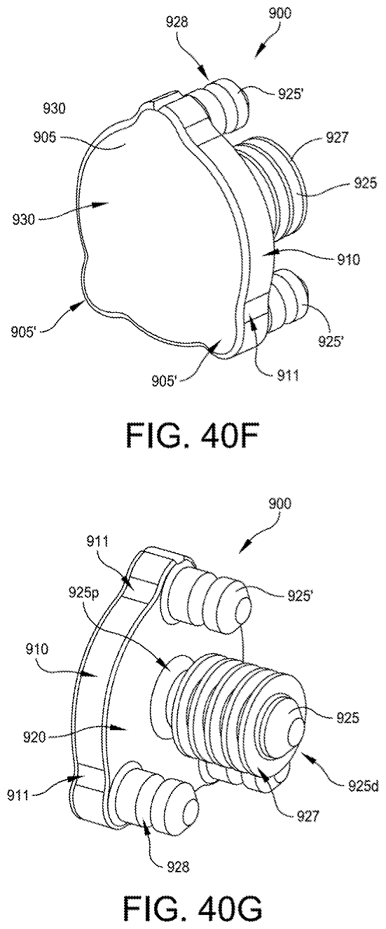

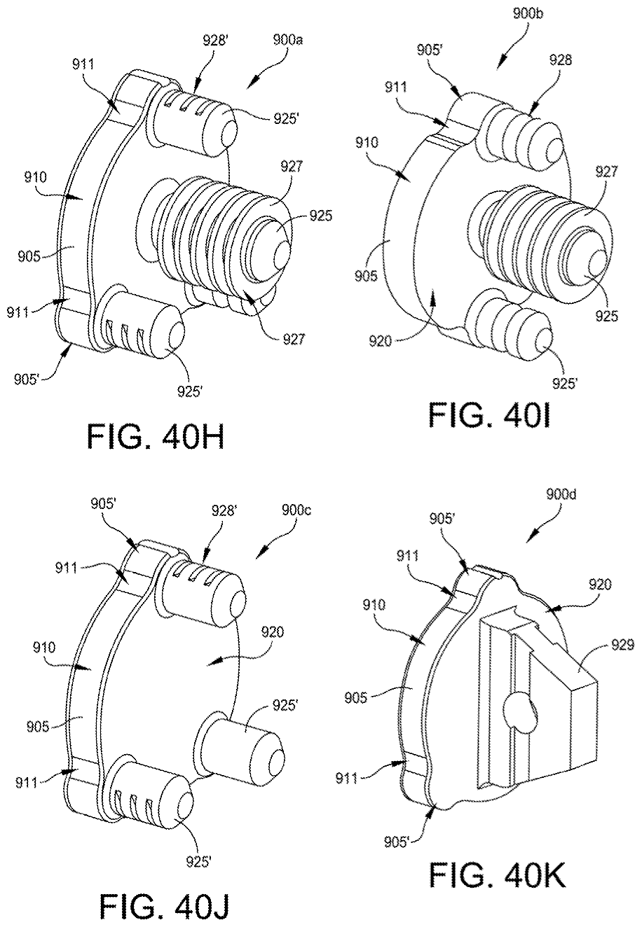

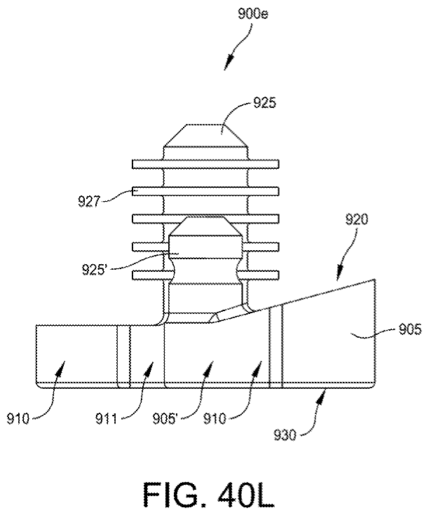

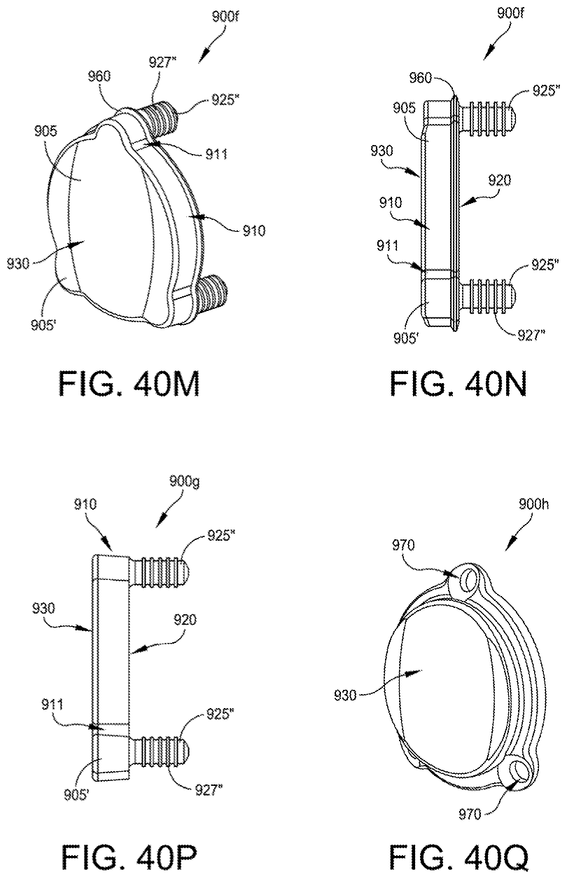

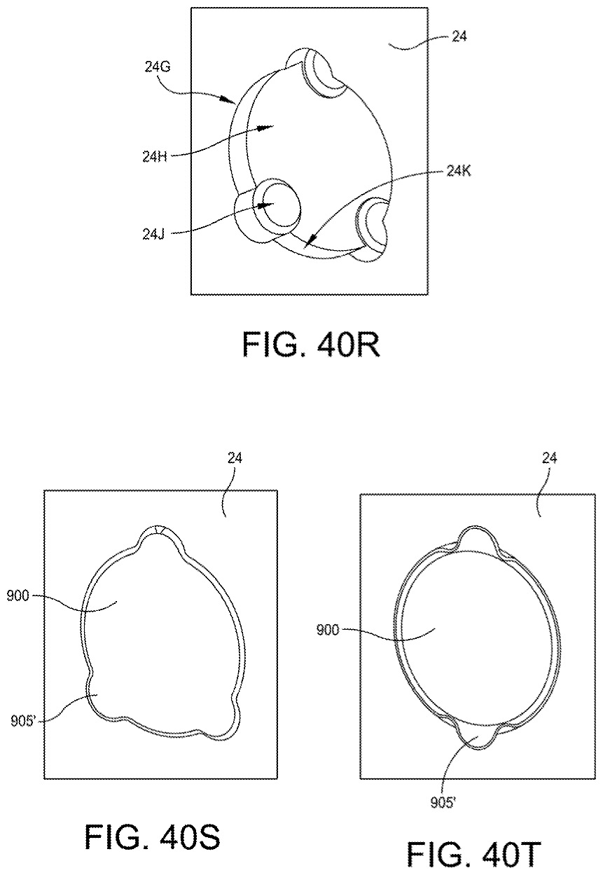

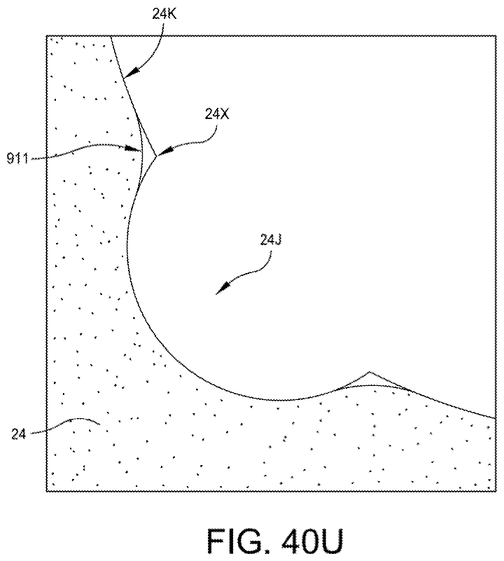

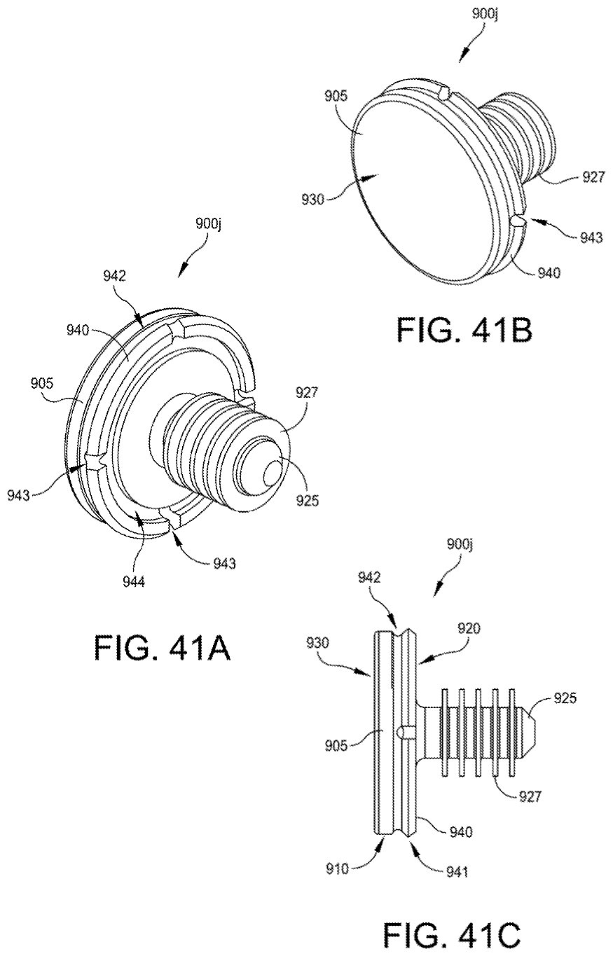

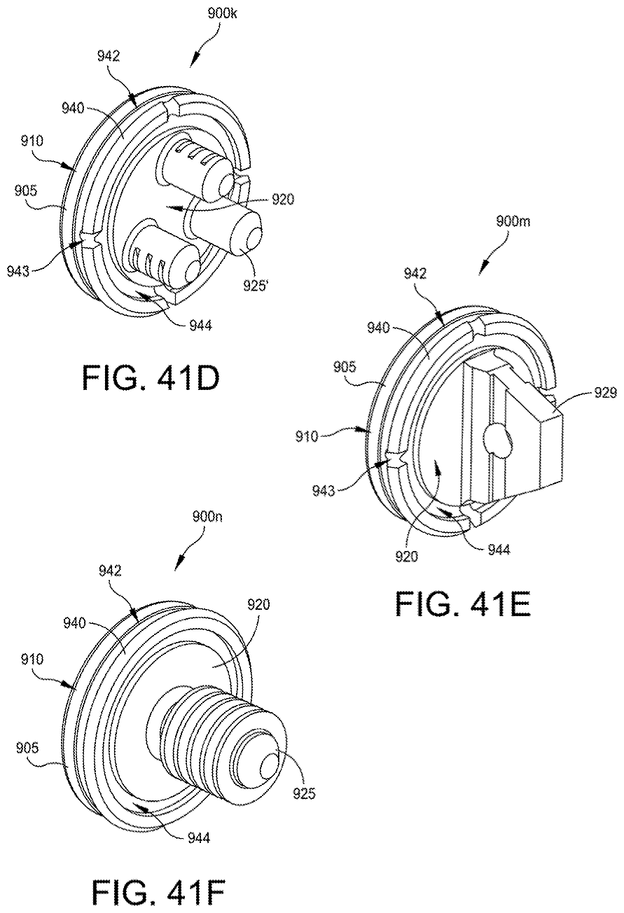

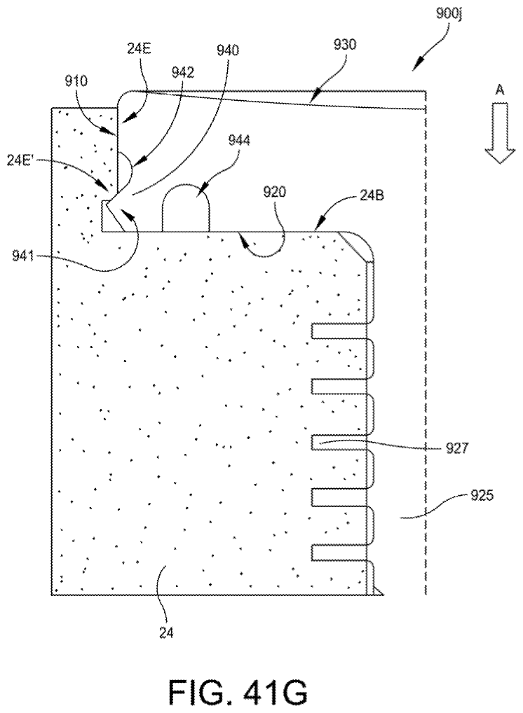

Referring to FIGS. 40A-41F, embodiments of inlay glenoid implants configured to provide improved fixation to glenoid that are integrally formed of a high modulus polymer material, such as UHMWPE or PEEK, are disclosed. These implants will be referred to herein as “all-polymer” implants. The all-polymer glenoid implants can comprise one or more peripheral fixation feature that is provided along the periphery (or circumference) of the implant body or even extend beyond the outer periphery of the implant body. Such placement of the fixation features improves the implants' stability, especially lateral stability, and the quality of fixation.

In some embodiments, the one or more peripheral fixation features can be tapered outer side wall profile, that enable interference fitting along the periphery of the implant body. In other embodiments, the one or more peripheral fixation features can be one or more anchoring elements that extend from the bottom or base surface of the implant body, such as posts, pegs, finned anchors, etc., that are positioned at locations beyond the outer periphery of the implant body. These fixation features can engage the glenoid with mechanical interference fitting, partial interference fitting, no interference fitting, or any combination thereof. These fixation features can also be augmented with bone cement. The fixation features augmented with bone cement can be provided with or without cement pockets.

Additionally, the all-polymer glenoid implants can further comprise conventional anchoring elements that extend from the base surface of the implant body, such as, posts, pegs, finned anchors, keels, etc., in addition to the peripheral fixation features.

The outer profile of the all-polymer glenoid implant can be tapered (i.e. frustoconical), straight without a taper (i.e. perpendicular to the base surface (the face opposite from the articulation surface) where the base surface is flat), or have a lip that creates an interference with the bone. The bone cavity prepared for receiving the glenoid implant can have an undercut created by an instrument that will mate with the interfering lip.

According to some embodiments, the all-polymer glenoid implant can also comprise one or more through holes so that bone screw(s) can be used for additional fixation, if appropriate.

According to some embodiments, the base surface of the all-polymer glenoid implant can be flat, concave, or convex.

Shown in FIGS. 40A-40C is an embodiment of an all-polymer glenoid implant 800 that is configured to be implanted into a glenoid in an inlay configuration. The glenoid implant 800 comprises a substantially circular disk-like body 805 having an articulation surface 830, an anchoring base surface 820 on the opposite side, and a side wall 810 extending between the two. The glenoid implant 800 can be configured to have a shape that maximizes the articulation surface of the glenoid 24 that is replaced by the glenoid implant 800. The articulation surface 830 is contoured to replicate the natural articulation surface of the glenoid 24.

For its peripheral fixation feature, the side wall 810 preferably has a tapered profile, that enables interference fitting along the periphery of the implant body when the implant 800 is implanted into a recess prepared in the glenoid 24. The taper of the side wall 810 is such that the articulation surface 830 is larger in diameter than the anchoring base surface 820. Thus, the body 805 has a shallow frusto-conical shape. The interference fitting provided by the tapered side wall 810 is a fixation feature that extends radially outward beyond the periphery of the substantially circular shaped body 805.

The glenoid implant 800 comprises additional features that helps secure the implant 800 in the glenoid 24. For example, extending from the anchor surface 820 is at least one finned anchor 825 and a plurality of stabilizing posts 825′. The articulation surface 830 is generally concave and is configured to engage a humeral head.

The finned anchor 825 and the stabilizing posts 825′ of the glenoid implant 800 extend from the base surface 820 and secure the glenoid component 800 to the glenoid 24. The stabilizing posts 825′ are positioned radially outward from the finned anchor 825 so that the stabilizing posts 825′ are located along the peripheral region of the glenoid implant 800 and offer stability to the glenoid implant 800 after implantation. In the illustrated example embodiment, the finned anchor 825 and the stabilizing posts 825′ extend substantially perpendicularly from the base surface 820. In some embodiments, the finned anchor 825 can extend at various other angles relative to the base surface 820. The finned anchor 825 is preferably positioned substantially at the center of the base surface 820 and is in the form of a cylindrical shaft having a proximal end 825p and a distal end 825d. (See FIG. 40C). The finned anchor 825 is attached to the base surface 820 at the proximal end 825p and tapers at the distal end 825d to facilitate insertion of the finned anchor 825 into an anchor receiving hole prepared in the glenoid 24. In one embodiment, the distal end 825d of the finned anchor 825 includes a conical tip or other shape that facilitates insertion into glenoid 24, with or without a pre-drilled hole. In the example shown in FIGS. 40A-40C, the distal end 825d has a conical tip.

In the illustrated example, the finned anchor 825 comprises a substantially constant diameter and further comprises a plurality of fins 827 that extend radially outward. The each of the fins 827 are spaced apart from each other along the length of the finned anchor 825. The fins 827 can be equally spaced or the spacing can be varied if desired.

The fins 827 are flexible and are configured to bend or deform when force is exerted against them. Deformation of the fins 827 can be plastic or elastic. In some embodiments, the fins 827 are formulated to deform plastically upon insertion into the glenoid 24 and assume a generally curved configuration once implanted. In some embodiments, the fins 827 are formulated to deform elastically upon insertion into the glenoid 24 and constantly exert some amount of force against the surrounding bone once implanted as the fins try to return to their un-deformed configuration.

In some embodiments, the finned anchor 825 and its fins 827 can be integrally formed with the body 805. For example, the glenoid implant 800 can be molded as a single unitary structure or machined from a monolithic piece of polymer material. In other embodiments, the finned anchor 825 and the body 805 are separate components. In an alternate embodiment, the body 805 can be molded from a first material while the finned anchor 825 and its fins 827 are molded from a second material. In this embodiment, the second material preferably has a higher stiffness than the first material.

The stabilizing posts 825′ prevent the glenoid implant 800 from moving relative to the glenoid 24 once the implant 800 is implanted in the glenoid 24. The stabilizing posts 825′ preferably extend substantially perpendicular to the base surface 820 of the implant 800. Each of the stabilizing posts 825′ includes a body having a proximal end 825p′ and a distal end 825d′. Each of the body of the stabilizing posts 825′ is attached at its proximal end 825p′ to the base surface 820 of the implant body 805. The stabilizing posts 825′ can also include an indent or a series of indents to accept and lock in bone cement, maintaining the stabilizing posts 825′ in position.

The stabilizing posts 825′ are preferably shorter than the finned anchor 825. Similar to the distal end 825d of the finned anchor 825, the distal ends 825′d of the stabilizing posts 825′ can also be tapered to facilitate insertion of the stabilizing posts 825′ into holes prepared in the glenoid 24. In some embodiments, the distal ends 825′d of the stabilizing posts 825′ have a conical tip, or other shape that facilitates insertion into the glenoid 24, with or without pre-drilled hole.

The stabilizing posts 825′ can be arranged in any configuration on the base surface 820. In one embodiment, the stabilizing posts 825′ positioned such that one of the stabilizing posts 825′ is positioned farther from the finned anchor 825 than the other stabilizing posts 825′. In another embodiment, the stabilizing posts 825′ are positioned around the finned anchor 825 along a periphery of the substantially equidistant from the finned anchor 825 and each adjacent stabilizing posts 825′.

The structure and function of the finned anchor 825 are similar to those of the similar anchor described in U.S. Pat. No. 10,524,922, the disclosure of which is incorporated herein by reference.

Referring to FIGS. 40D-40E, to receive the glenoid implant 800, a recess 24A sized and shaped appropriately for the glenoid implant 800 is reamed into a glenoid 24. The recess comprises a bottom surface 24B into which a hole 24C is drilled to receive the anchor 825 and additional holes 24D are drilled to receive the stabilizing posts 825′. The holes 24C and 24D have the appropriate diameter and depth to receive the corresponding finned anchor 825 or the stabilizing post 825′. The recess 24A has a sidewall 24E that is tapered to match the taper of the side wall 810 of the glenoid implant 800. FIG. 40E shows the glenoid implant 800 seated in the recess 24A.

In some embodiments, each of the stabilizing posts 825′ can comprise one or more bone cement pockets 828. When the glenoid implant 800 is being secured into the corresponding recess 24A prepared in the glenoid 24, bone cement can be used to enhance the securement of the implant. Each of the pockets 828 can hold an amount of bone cement.

Shown in FIGS. 40F-40G is an all-polymer glenoid implant 900 configured for an inlay implantation into a glenoid according to another embodiment. The glenoid implant 900 comprises a body 905 comprising an articulation surface 930, a base surface 920 on the opposite side, and a side wall 910 extending between the two. The glenoid implant 900 can be configured to have a shape that maximizes the articulation surface of the glenoid 24 that is replaced by the glenoid implant 900. The articulation surface 930 is contoured to replicate the natural articulation surface of the glenoid 24.

The side wall 910 can have a tapered profile, that enables interference fitting along the periphery of the implant body when the implant 900 is implanted into a recess prepared in the glenoid 24. The taper of the side wall 910 is such that the articulation surface 930 is larger in diameter than the base surface 920.

In this embodiment, the body 905 has a plurality of protruding portions 905′ that extend radially outward beyond the outer periphery (or circumference) of the substantially circular shape of the body 905 and a fixation feature such as a stabilizing post 925′ is provided on each of the protruding portions 905′ extending from the base surface. Because the protruding portions 905′ allow placement of the fixation features outside the periphery of the body 905, this configuration enhances the lateral stability of the glenoid implant 900 in the bone and help mitigate rocking of the implant.

The glenoid implant 900 can further comprise one or more additional non-peripheral fixation features that helps secure the implant 900 in the glenoid 24. The non-peripheral fixation features can comprise any one of a post, a finned anchor, or a keel, etc. For example, in the illustrated example glenoid implant 900, extending from the anchor surface 920 is a finned anchor 925. The articulation surface 930 is generally concave and is configured to engage a humeral head.

The finned anchor 925 and the stabilizing posts 925′ of the glenoid implant 900 extend from the base surface 920 and secure the glenoid component 900 to the glenoid 24. The stabilizing posts 925′ are positioned radially outward from the finned anchor 925 so that the stabilizing posts 925′ are located along the peripheral region of the glenoid implant 900 and offer stability to the glenoid implant 900 after implantation.

In the illustrated example embodiment, the finned anchor 925 and the stabilizing posts 925′ extend substantially perpendicularly from the base surface 920. In some embodiments, the finned anchor 925 can extend at various other angles relative to the base surface 920. The finned anchor 925 is preferably positioned substantially at the center of the base surface 920 and is in the form of a cylindrical shaft having a proximal end 925p and a distal end 925d. (See FIG. 40G). The finned anchor 925 is attached to the base surface 920 at the proximal end 925p and tapers at the distal end 925d to facilitate insertion of the finned anchor 925 into an anchor receiving hole prepared in the glenoid 24. In one embodiment, the distal end 925d of the finned anchor 925 includes a conical tip or other shape that facilitates insertion into glenoid 24, with or without a pre-drilled hole. In the example shown in FIGS. 40F-40G, the distal end 925d has a conical tip.

In the illustrated example, the finned anchor 925 comprises a substantially constant diameter and further comprises a plurality of fins 927 that extend radially outward. The each of the fins 927 are spaced apart from each other along the length of the finned anchor 925. The fins 927 can be equally spaced or the spacing can be varied if desired.

The fins 927 are flexible and are configured to bend or deform when force is exerted against them. Deformation of the fins 927 can be plastic or elastic. In some embodiments, the fins 927 are formulated to deform plastically upon insertion into the glenoid 24 and assume a generally curved configuration once implanted. In some embodiments, the fins 927 are formulated to deform elastically upon insertion into the glenoid 24 and constantly exert some amount of force against the surrounding bone once implanted as the fins try to return to their un-deformed configuration.

In some embodiments, the finned anchor 925 and its fins 927 can be integrally formed with the body 905. For example, the glenoid implant 900 can be molded as a single unitary structure or machined from a monolithic piece of polymer material. In other embodiments, the finned anchor 925 and the body 905 are separate components. In an alternate embodiment, the body 905 can be molded from a first material while the finned anchor 925 and its fins 927 are molded from a second material. In this embodiment, the second material preferably has a higher stiffness than the first material.

The functions of the finned anchor 925 and the stabilizing posts 925′ are similar to those of the finned anchor 825 and the stabilizing posts 825′ of the glenoid implant 800 except that the stabilizing posts 925′ are located so that they are positioned beyond the periphery of the substantially circular body 905 to further enhance the lateral stability of the implant 900 when implanted in a glenoid 24.

In some embodiments, each of the stabilizing posts 925′ can comprise one or more bone cement pockets 928. When the glenoid implant 900 is being secured into the corresponding recess prepared in the glenoid 24, bone cement can be used to enhance the securement of the implant. Each of the pockets 928 can hold an amount of bone cement.

In some embodiments, the glenoid implant 900 can be provided in a variety of configurations to have different types and numbers of fixation elements. For example, the implant 900 can have just one type of fixation elements extending from the anchoring surface 920 (i.e. finned anchor 925, the stabilizing posts 925′, keel 929, etc.) as one or more fixation elements provided. In other embodiments, the implant 900 can have different bone cement pockets, or even different types of anchoring elements such as keel. For example, FIG. 40H shows an all-polymer glenoid implant embodiment 900a in which the stabilizing posts 925′ have one or more bone cement pockets 928′ that are slots cut into the stabilizing posts 925′. FIG. 40I shows an all-polymer glenoid implant embodiment 900b that comprises two stabilizing posts 925′ that are located 180° apart from one another. Also, the cement pocket 928 on the stabilizing posts 925′ are grooves. FIG. 40J shows an all-polymer glenoid implant embodiment 900c that has the same three stabilizing posts 925′ as those in the embodiment of FIG. 40H but does not have any finned anchor 925. In the all-polymer glenoid implant embodiment 900c, the base surface 920 can be substantially flat or can have a slightly convex or concave contour. The benefits of a convex contoured base surface 920 can be that such surface is more conforming to natural glenoid articulation, thus requiring less subchondral (good) bone reaming. Convex surface also provides larger contact surface area than a flat base surface. The benefits of a concave contoured base surface 920 is that it may improve the stability of the implant by reversing the “soap-dish” effect that can cause lateral slippage of the implant under peripheral loading conditions. Furthermore, a concave back surface allows the periphery of the implant to be situated deeper in the bone while preserving cortical bone thickness in the center where the forces are the greatest and the primary non-peripheral fixation feature is located.

FIG. 40K shows an all-polymer glenoid implant embodiment 900d that has a keel 929 rather than finned anchors 925 or stabilization posts 925′. The keel 929 and its variants are similar to the keel for a glenoid component described in U.S. Pat. No. 8,080,063, the contents of which are incorporated herein by reference. In some embodiments, the glenoid implant 900 can be configured with any combination of the fixation features described herein as appropriate.

FIG. 40L shows an all-polymer glenoid implant embodiment 900e where the base surface 920 of the implant can be contoured in any variety of ways to accommodate the condition of the glenoid 24. In the illustrated example, the base surface 920 is slanted on one side of the implant to form a wedge-shaped profile for the body 905 of the implant 900.

FIGS. 40M and 40N show an all-polymer glenoid implant embodiment 900f that comprises a plurality of protruding portions 905′ that are provided with mini-finned anchors 925″ extending from the base surface 920 as one or more peripheral fixation features. The glenoid implant 900f further comprises a protruding lip 960 along the periphery of the base surface 920 side of the body 905 as additional peripheral fixation feature. The protruding lip 960 engages the side wall 24K (see the recess 24G in FIG. 40M) of the recess prepared into the glenoid 24 and creates an interference fit to help secure the implant 900f in the glenoid 24.

This shape is intended to control the translation of the mating humeral head, allowing it to only translate across the surface in one linear vector. For example, depending on the orientation of the “swept” geometry the glenoid could easily transvers the glenoid surface along an inferior-superior trajectory but meets with resistance to traversing the glenoid in an anterior-posterior motion.

FIG. 40P is a side view of an all-polymer glenoid implant embodiment 900g that comprises a tapered side wall 910 and a plurality of protruding portions 905′ that are provided with mini-finned anchors 925″ extending from the base surface 920 as the one or more peripheral fixation features. This glenoid implant 900g does not have any non-peripheral fixation features.

FIG. 40Q shows an all-polymer glenoid implant embodiment 900h in which a plurality of screw holes 970 are the peripheral fixation feature and does not have any additional non-peripheral fixation features such as posts, finned anchors, or keel. The glenoid implant 900h is secured to a glenoid using bone screws. In some other embodiments, the glenoid implant can have other fixation features in addition to the screw holes.

FIG. 40R is an illustration of a glenoid 24 that has been reamed to form a recess 24G that is sized and shaped appropriately for receiving the glenoid implant 900 example shown in FIG. 40J. The recess 24G comprises a bottom surface 2411, side wall 24K and holes 24J that have been drilled along the periphery of the recess 24G for receiving the stabilizing posts 925′ and to accommodate and engage the protruding portions 905′ that extend radially outward beyond the outer periphery of the substantially circular shape of the implant body 905.

FIG. 40S is an illustration of the implant 900 of FIG. 40J that has been implanted into the recess 24G. FIG. 40T is an illustration of the implant 900 of FIG. 40I that has been implanted into a recess, similar to the recess 24G, that is shaped and sized for the implant 900 of FIG. 40I.

As can be seen in FIGS. 40F-40K, in some embodiments of the glenoid implant 900, the transition region 911 between the protruding portion 905′ and the remainder of the side wall 910 portion is a curved surface. The outline of the recess 24G and the holes 24J formed in the glenoid 24 are shaped to form an interference fitting engagement with the curved transition region 911 of the glenoid implant 900. This is illustrated in FIG. 40U. FIG. 40U shows the portion of the glenoid 24 around one of the holes 24J that has been prepared in the glenoid 24. Where the side wall 24K of the recess 24G meets the hole 24J is a cusp 24X. This cusp 24X and the curved transition region 911 of the glenoid implant 900 overlap each other and thus form an interference fit. In FIG. 40U, the outline of the transition region 911 of the glenoid implant 900 is shown overlapping with the cusp 24X which enables the interference fit.

FIGS. 41A-41C show another embodiment of all-polymer glenoid implant 900j. The glenoid implant 900j comprises a locking rim structure 940 as its peripheral fixation feature that secures the implant 900j in a recess prepared in a glenoid 24 by pushing the body 905 of the implant into the recess. The glenoid implant 900j further comprises a finned anchor 925 as the one or more additional fixation feature. As can be better seen in the side view FIG. 41C, the locking rim 940 is defined by an annular groove 942 formed into the outer side wall 910 of the substantially circular body 905 and an undercut groove 944 formed into the base surface 920 of the body 905. The locking rim 940 has a larger diameter than the substantially circular body 905 and comprises an edge 941 that protrudes radially outward from the body 905. The engagement between the locking rim 940 and the recess prepared in a glenoid 24 can be seen in detail in FIG. 41G. FIG. 41G is a partial cross-sectional view of the all-polymer glenoid implant 900j that is implanted into a recess prepared in a glenoid 24. The cross-section shows the structure of the locking rim 940. The annular groove 942 and the undercut groove 944 define the locking rim 940. The edge 941 of the locking rim 940 protrudes radially outward beyond the side wall 910. The recess reamed into the glenoid is similar to the recess 24A shown in FIG. 40D and has a side wall 24E and a bottom surface 24B. To receive the glenoid implant 900j, the bottom portion of the side wall 24E of the recess is reamed further radially outward with an undercut thus forming an overhang 24E′. Because the edge 941 of the locking rim 940 protrudes radially outward, the rim 940 has a larger diameter than the diameter of the recess formed by the side wall 24E. Thus, as the glenoid implant 900j is being pushed into the recess in the glenoid 24 in the direction noted by the arrow A, the locking rim 940 elastically bends radially inward until the edge 941 clears the overhang 24E′. Once the edge 941 clears the overhang 24E′, the locking rim 940 snaps back outward into the configuration shown in FIG. 41G where the edge 941 and the overhang 24E′ creates a mechanical lock that secures the glenoid implant 900j in place. As the glenoid implant 900j is an all-polymer implant, the polymer material allows the locking rim 940 to elastically bend as described. The modulus of elasticity of the locking rim 940 can be tuned by selecting appropriate polymer formulation.

Alternatively, in some embodiments, the side wall 24E of the recess in the glenoid 24 can be simply straight without any undercut and the edge 941 of the locking rim 940 simply creates an interference fit with the reamed recess.

According to some embodiments, the locking rim 940 can be further configured with a plurality of compression relief cutouts 943 as shown. The compression relief cutouts 943 divides the locking rim 940 into multiple segments as shown. Preferably, the compression relief cutouts 943 are located at positions that radially symmetric so that the locking rim 940 is divided into equal-sized segments thereby providing radially symmetric compression relief as the glenoid implant 900f is pushed into the recess in the glenoid 24.

FIGS. 41D-41F show examples of additional embodiments of the all-polymer glenoid implant comprising the locking rim 940 feature as the peripheral fixation feature but are configured with one of a variety of other possible fixation features that extend from the base surface 920. FIG. 41D shows an all-polymer glenoid implant embodiment 900k that comprises the locking rim 940 like the glenoid implant 900j but has three stabilizing posts 925′ extending from the base surface 920 as the one or more additional fixation features. FIG. 41E shows an all-polymer glenoid implant embodiment 900m that also comprises the locking rim 940 like the glenoid implant 900j but has a keel 929 extending from the base surface 920 as the one or more additional fixation features. FIG. 41F shows an all-polymer glenoid implant embodiment 900n that also comprises the locking rim 940 like the glenoid implant 900j but the locking rim 940 does not have the compression relief cutouts.

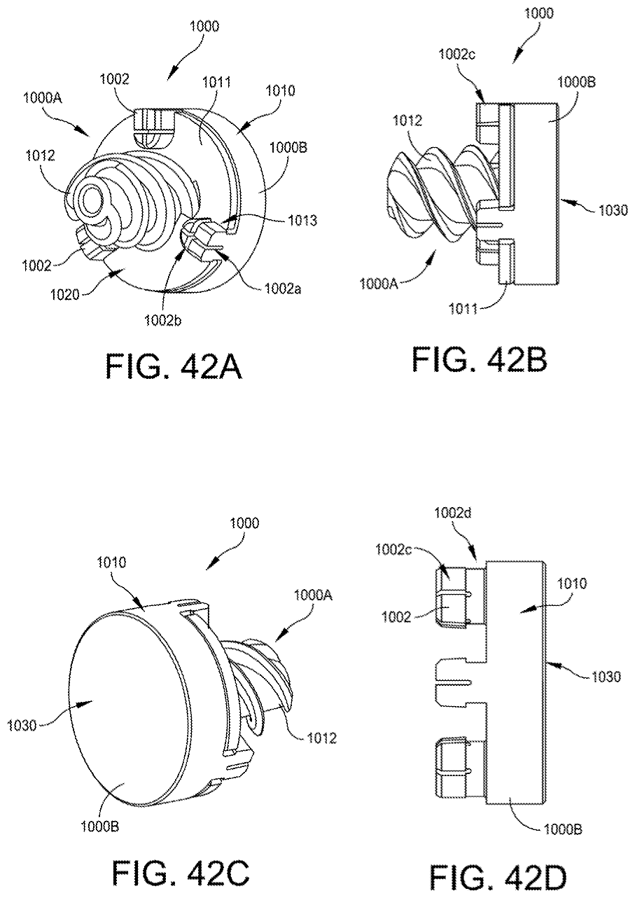

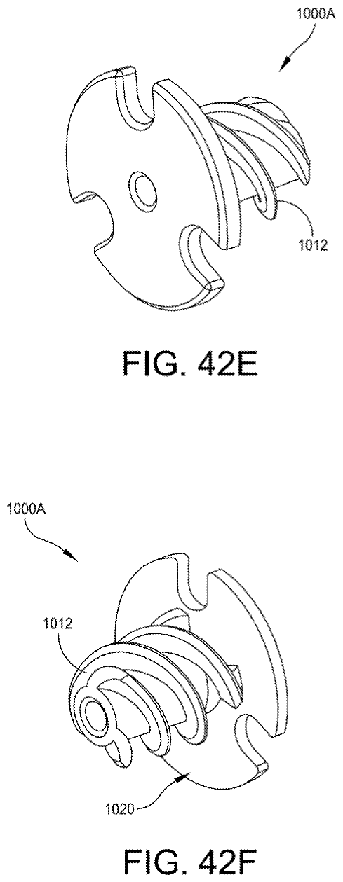

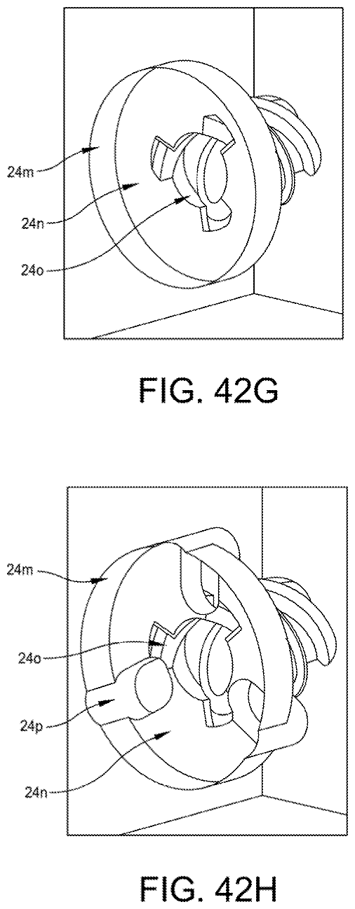

FIGS. 42A-42D show a metal-backed glenoid implant embodiment 1000. The metal-backed glenoid implant comprises two-piece construction: a metal anchor 1000A, and a polymer insert 1000B that are configured to lock into each other during the implantation process. The polymer insert 1000B can be made of high modulus polymer material, such as UHMWPE or PEEK. The metal anchor 1000A comprises a circular plate portion 1011 comprising two faces and a threaded screw portion 1012 extending from the center of one of the two faces that is the bone-facing base surface 1020. The face that is opposite of the base surface 1020 is the one that receives the polymer insert 1000B. The plate portion 1011 is configured with a plurality of cutouts or notches 1013 along the periphery of the plate portion 1011. The polymer insert 1000B comprises an articulation surface 1030 and further comprises a plurality of tabs 1002 extending from the polymer insert 1000B on the side opposite from the articulation surface 1030. The tabs 1002 are configured to snap into the corresponding cutouts 1013. The number and location of the tabs 1002 match the number and location of the plurality of cutouts 1013 on the plate portion 1011. The polymer insert 1000B and the metal anchor 1000A lock into each other by aligning and inserting the tabs 1002 through the cutouts 1013. FIGS. 42E-42F are illustrations of the metal anchor 1000A without the polymer insert 1000B.

During the implantation process, the metal anchor 1000A is first screwed into a bone 24 that is prepared with a recess 24m (See FIG. 42H) that has a tapped threaded hole 24n. Once the metal anchor 1000A is threaded in place in the recess 24m, the polymer insert 1000B is snapped onto the plate portion 1011 of the metal anchor 1000A by first aligning the plurality of tabs with the cutouts 1013 and pushing the polymer insert 1000B toward the plate portion 1011 until the tabs 1002 are fully inserted through the cutouts 1013 and lock. Each of the tabs 1002 comprise one or more compression relieving slots 1002a, 1002b and the leading end 1002c of the tabs 1002 are larger than the cutouts 1013. This allows the tabs 1002 to compress as they get squeezed into the cutouts 1013 then spring back to their resting configuration once the polymer insert 1000B is fully engaged with the metal anchor 1000A. FIG. 42D is a side view of the polymer insert 1000B. Each of the tabs 1002 has a leading end 1002c that is somewhat larger than the opening provided by the cutouts 1013 and a neck portion 1002d that is sized to match the size of the opening provided by the cutouts 1013. Thus, when the tabs 1002 are inserted into the cutouts 1013 the compression relieving slots 1002a, 1002b allow the leading end 1002c on each of the tabs 1002 to compress ad allow the tabs 1002 to fit through the cutouts 1013. Once the cutouts 1013 get past the leading end 1002c and engage the neck portion 1002d, the leading end 1002c decompresses back to its resting state creating an interference fit between the leading end 1002c and the plate portion 1011 and hold the polymer insert 1000B and the metal anchor 1000A together. As can be seen in FIGS. 42A-42C, when the metal anchor 1000A and the polymer insert 1000B are assembled together, the leading ends 1002c of the tabs 1002 protrude from the base surface 1020 of the metal anchor 1000A.

FIGS. 42G-42H are illustrations showing an example of a circular recess 24m that would be prepared into a bone for receiving the metal-backed glenoid implant 1000. FIG. 42G-42H show a graphical rendering of the 3-dimensional form of just the surface of the bone after the recess 24m is formed and the bulk bone material is removed. Referring back to FIG. 42G, the circular recess 24m has a bottom surface 24n. After the circular recess 24m is reamed into the bone, a threaded hole 24o is tapped into the bottom surface 24n for receiving the threaded screw portion 1012 of the metal anchor 1000A. Referring to FIG. 42H, next, a plurality of deeper recesses 24p are reamed or drilled out in an arrangement that match the arrangement of the tabs 1002. These deeper recesses 24p provide clearance space for the tabs 1002 that protrude from the base surface 1020 of the metal anchor 1000A when the metal-backed glenoid implant 1000 is implanted into the bone. Once the recess 24m is fully prepared as shown in FIG. 42H, because of the threading action that is required to implant the glenoid implant 1000, the implant procedure involves two steps. First the metal anchor 1000A is threaded into the recess 24m until the metal anchor 1000A is fully seated and the base surface 1020 of the metal anchor 1000A is in contact with the bottom surface 24n of the recess 24m. At this point, the metal anchor 1000A is seated at the bottom of the recess 24m. Next, the tabs 1002 of the polymer insert 1000B are aligned with the cutouts 1013 in the metal anchor 1000A and the polymer insert 1000B is pushed into the recess 24m until the leading ends 1002c of the tabs 1002 are pushed through the cutouts 1013 and snapped in place. The deeper recesses 24p provide the clearance for the leading ends 1002c of the tabs 1002. Additionally, because the leading ends 1002c of the tabs 1002 are sitting in the deeper recesses 24p they prevent the implant 1000 from turning so that the implant 1000 cannot back out by unscrewing.

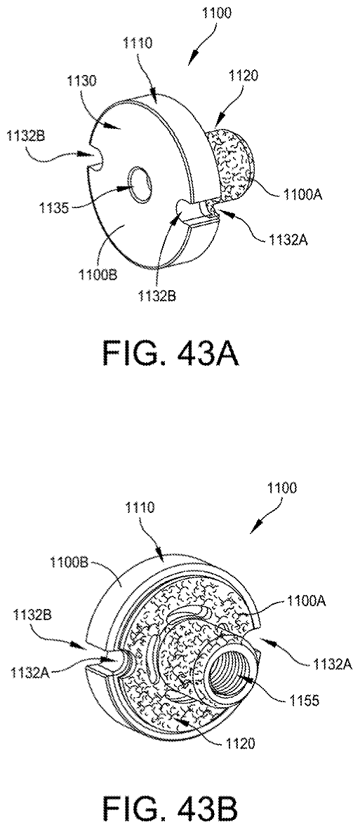

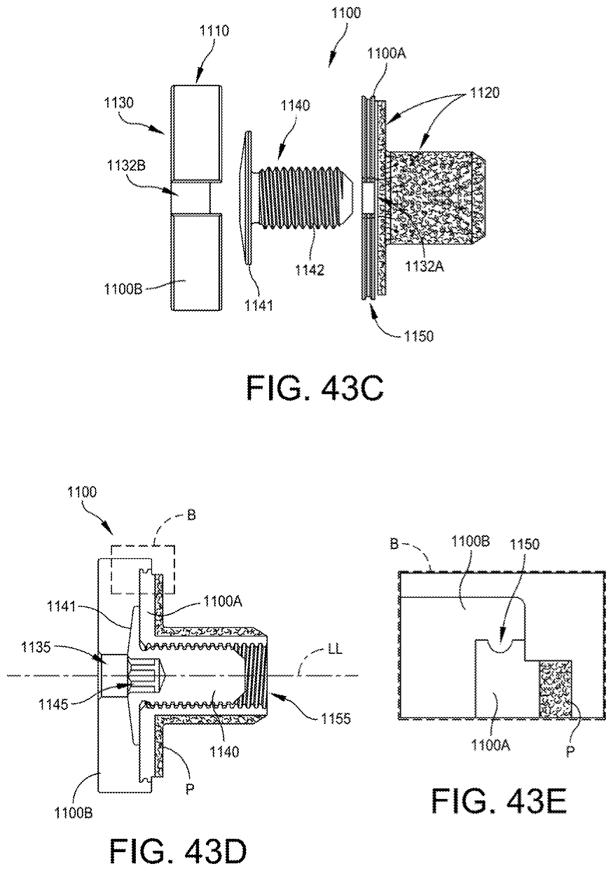

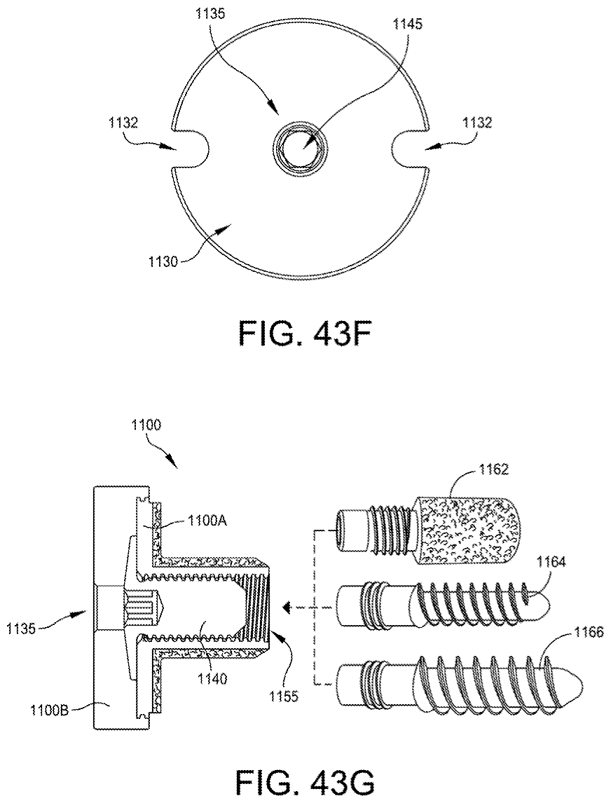

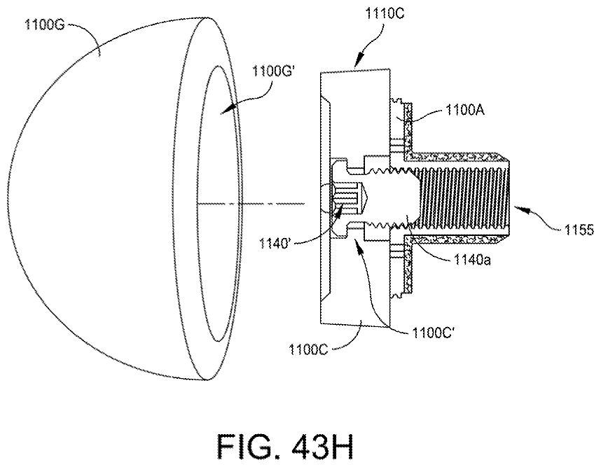

FIGS. 43A-43B show a porous metal-backed glenoid implant embodiment 1100. The glenoid implant embodiment 1100 comprises a polymer insert 1100B, providing the articulation surface 1130, that is overmolded directly onto a metal baseplate 1100A. The polymer insert 1100B can be made of high modulus polymer material, such as UHMWPE or PEEK. The articulation surface 1130 for engaging a humeral head (anatomical one or a prosthetic one). In the overmolded structure for the implant 1100 shown in FIGS. 43A-43B, the metal baseplate 1100A provides the bone-contacting base surface 1120. In some embodiments, the bone-contacting base surface 1120 is coated with a porous trabecular metal material, such as Wright Medical Technology's ADAPTIS™ that can promote bone tissue ingrowth to enhance bonding of the glenoid implant 1100 to glenoid after implantation. FIG. 43C identifies the bone-contacting base surface 1120. The cross-sectional view of the implant 1100 in FIG. 43D shows the porous trabecular metal coating P on the base surface 1120.

In some embodiments, the side wall 1110 of the polymer insert 1100B can be tapered for enhanced peripheral fixation.

Although the polymer insert 1100B is overmolded onto the metal baseplate 1100A, the implant 1100 is an assembly that is configured to be able to remove the polymer insert 1100B from the metal baseplate 1100A if necessary. The exploded view of the implant 1100 in FIG. 43C shows that the implant 1100 comprises three components: the baseplate 1100A, a removal wedge screw 1140, and the overmolded polymer insert 1100B. The removal wedge screw 1140 is assembled into the metal baseplate 1100A before the polymer insert 1100B is overmolded onto the metal baseplate 1100A. The removal wedge screw 1140 threads into the threaded hole 1155 that extends through the metal baseplate 1100A along the longitudinal axis LL of the metal baseplate 1100A. The removal wedge screw 1140 comprises a head portion 1141 and a threaded stem portion 1142. Then, the polymer insert 1100B is overmolded over the baseplate 1100A and the head portion 1141 of the removal wedge screw 1140. The head portion 1141 has a diameter that covers a substantial portion of the metal baseplate 1100A such that substantial portion of the surface area of the metal baseplate that is covered by the overmolded polymer insert 1100B is the head portion 1141. As will be described below, this configuration allows the removal of the overmolded polymer insert 1100B using the removal wedge screw 1140. As shown in the cross-sectional view in FIG. 43D, the removal wedge screw 1140 comprises a tool-engaging socket 1145 at the center of its head portion 1141 that can be used to screw or unscrew the removal wedge screw 1140 with the threaded hole 1155. The tool-engaging socket 1145 can be configured to mate with one of a variety of known types of screwdrivers. The overmolded polymer insert 1100B is provided with an access hole 1135 at the center of the insert 1100B providing access to the tool-engaging socket 1145. In FIG. 43F, which is a view looking straight on to the articulation surface 1130 of the polymer insert 1100B, the tool-engaging socket 1145 can be seen through the access hole 1135. In this example, the tool-engaging socket 1145 is a type that accepts a hexagonal screwdriver tip. By unscrewing the removal wedge screw 1140 out of the threaded hole 1155 of the metal baseplate 1100A, the head portion 1141 of the removal wedge screw 1140 will lift the overmolded polymer insert 1100B off from the baseplate 1100A for removal. To further enable this removal procedure, both the baseplate 1100A and the polymer insert 1100B are each configured with a pair of slots 1132A and 1132B, respective, that are aligned with each other. These slots 1132A, 1132B are used for providing counter-torque when removing the removal wedge screw 1140. When unscrewing the removal wedge screw 1140, an appropriate tool can be inserted into the pair of slots 1132A, 1132B to hold the baseplate 1100A and the polymer insert 1100B in place and keep them from turning with the removal wedge screw 1140.

FIG. 43E shows a detailed view of the region B in FIG. 43D showing the overmolded polymer insert 1100B bonding the metal baseplate 1100A. In some embodiments, to enhance the mechanical integrity of the bonding between the two components, the baseplate 1100A can comprise a groove 1150 that extend along the periphery of the baseplate 1100A which results in a more convoluted mating interface between the two components that provides mechanically stronger bonding interface than a straight one, for example.

In some embodiments, different fixation options, such as, modular posts, modular screws, keel, etc. can be used with this porous metal-backed glenoid implant 1100. For example, a modular post 1162 or a modular screw 1164, 1166 can be threaded into the threaded hole 1155 as illustrated in FIG. 43G.

FIG. 43H shows an embodiment where the implant 1100 can be converted to a reverse construct shoulder implant. For this conversion, instead of the polymer insert 1100B, a taper boss 1100C that is configured to mate with a glenosphere 1100G is attached to the metal baseplate 1100A. In FIG. 43H, the metal baseplate 1100A and the taper boss 1100C are shown in cross-section. In some embodiments, the taper boss 1100C can be configured to be attached to the metal baseplate 1100A by a screw 1140a. The screw 1140a threads into the threaded hole 1155 of the metal baseplate 1100A as shown. The taper boss 1100C comprises a hole in the center for receiving the screw 1140a and the hole in the taper boss 1100C comprises a ledge 1100C′ that extends inward and catches the head of the screw 1140a. Thus, the head of the screw 1140a captures the taper boss 1100C between the head of the screw and the metal baseplate 1100A and secures the taper boss 1100C. The head of the screw is provided with a tool-engaging socket 1140a′ that is configured to mate with one of a variety of known types of screwdrivers. The taper boss 1100C has a sidewall 1110 that is tapered to engage the glenosphere 1100G via a Morse taper type locking connection. The glenosphere 1100G comprises a corresponding female taper surface 1100G′ that engages the male taper surface 1110. The taper boss 1100C can also be configured with screw holes (not shown) that align with the slots 1132A in the metal baseplate 1100A and can accept screws for enhanced fixation.

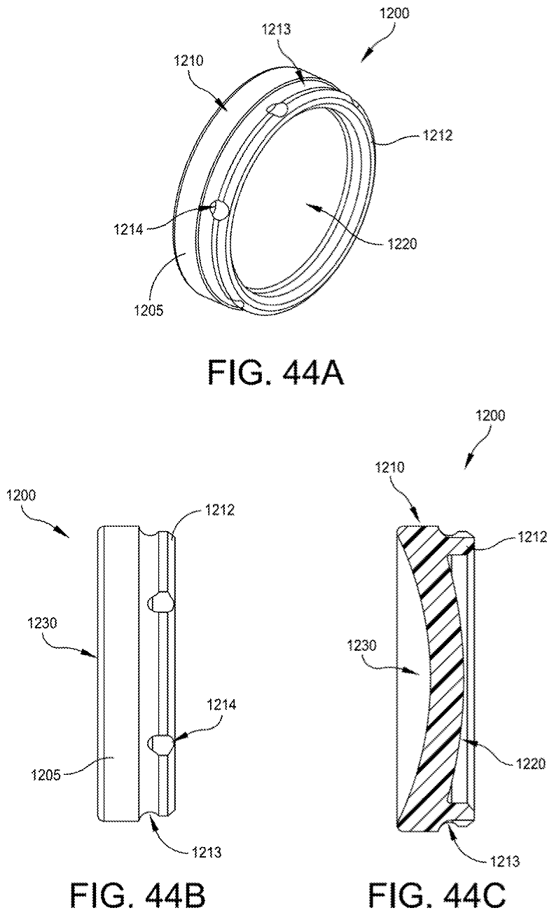

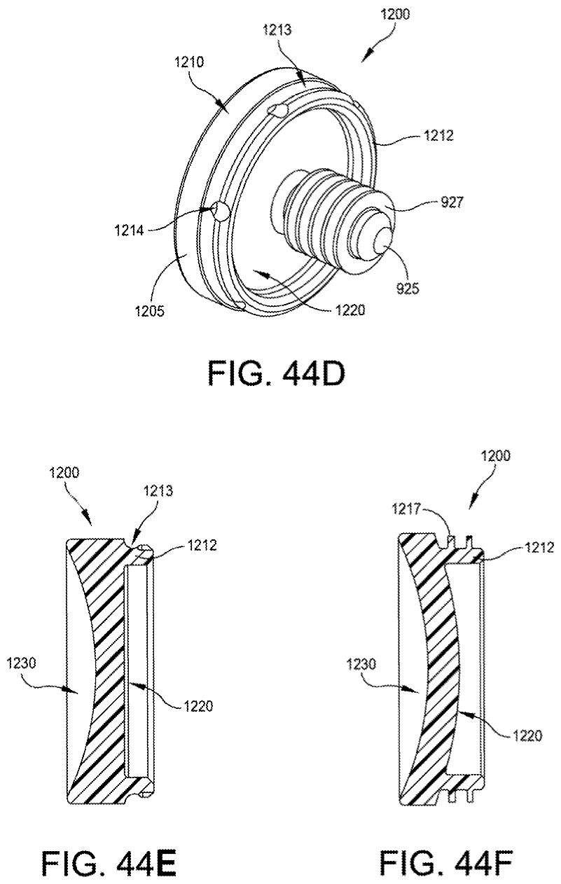

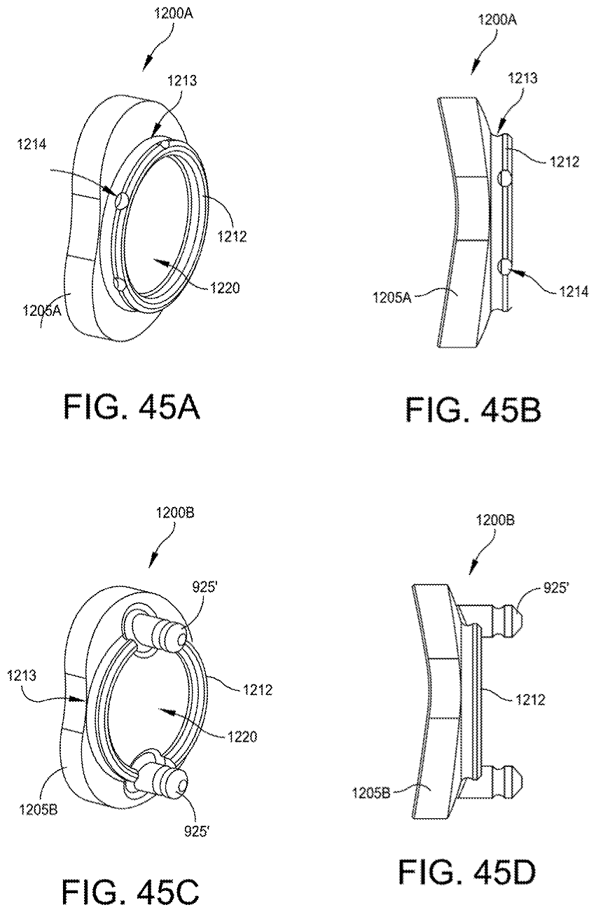

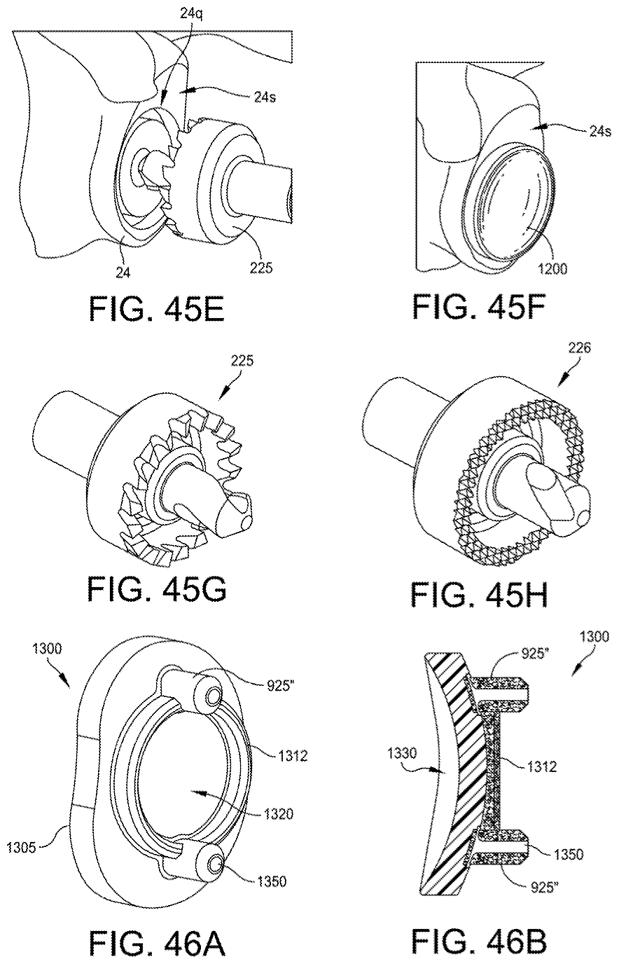

FIGS. 44A-45D show embodiments of glenoid implants employing peripheral ring fixation feature. Referring to FIG. 44A The glenoid implant 1200 comprises a substantially circular body 1205 comprising an articulation surface 1230 on one side and a bone-facing base surface 1220 on the opposite side. Around the periphery of the circular body 1205 is a side wall 1210 that extends between the two surfaces 1220 and 1230. Extending from the bone-facing base surface 1220 is a peripheral fixation feature that comprises a ring 1212 for engaging a glenoid. As shown in the cross-sectional view in FIG. 44C, in some embodiments, the articulation surface 1230 is contoured to replicate the anatomical articulation surface of the glenoid. In some embodiments, the articulation surface 1230 can have a spherical contour if necessary. The bone-facing base surface 1220 has a spherical contour to engage the glenoid that has been prepared with a complementary surface for receiving the implant 1200.

The glenoid implant 1200 further comprises a peripheral ring 1212 that extends from the periphery of the bone-facing surface 1220 and also engages the glenoid which has been prepared with an annular recess 24q (see FIG. 45E) for receiving the peripheral ring 1212. In this embodiment 1200 of the glenoid implant where the implant body 1205 has a substantially circular shape, the peripheral ring 1212 is an extension of the side wall 1210. The peripheral ring 1212 is configured to enhance the quality of fixation to the glenoid. The peripheral ring 1212 comprises a groove 1213 provided on the outer surface of the ring 1212 and extends around the periphery of the ring 1212. The groove 1213 serves the purpose of holding a quantify of bone cement along the periphery of the ring 1212 to bond to the glenoid. The peripheral ring 1212 can also comprise optional additional plurality of cement grooves 1214 provided along the outer surface of the ring 1212 adjacent to the groove 1213. The plurality of cement grooves 1214 are oriented axially. In preferred embodiments, the plurality of cement grooves 1214 are located in radially symmetric locations along the ring 1212. The application of the additional bone cement via the plurality of cement grooves 1214 is intended to prevent rotation of the glenoid implant 1200 after implantation.

In some embodiments, the glenoid implant 1200 can further comprise one or more additional fixation features extending from the base surface 1220. These additional fixation features can be any one of the fixation features such as posts, finned anchors, keels, etc. FIG. 44D shows an example of a glenoid implant 1200 comprising a finned anchor 925 extending from the center of the base surface 1220.

In some other embodiments, the base surface 1220 of the glenoid implant 1200 can be a flat surface as shown in the example cross-section shown in FIG. 44E. In some other embodiments, the outer surface of the peripheral ring 1212 is provided with flexible fins 1217 for cementless application as shown in the example cross-sectional view shown in FIG. 44F. The fins 1217 serve the same function as the fins 927 on the finned anchors 925.

FIGS. 45A-45B show another glenoid implant 1200A embodiment that comprises an implant body 1205A and a peripheral ring 1212 provided on the bone-facing base surface 1220 as a peripheral fixation element similar to the glenoid implant 1200 shown in FIG. 44A. The peripheral ring 1212 for the glenoid implant 1200A is the same structural features as the peripheral ring 1212 for the glenoid implant 1200, including all of the optional features. In the embodiment 1200A, however, the implant body 1205A is not circular but has a shape that mirrors the outline of an anatomical glenoid. Similar to the glenoid implant 1200, the glenoid implant 1200A can also be configured with one or more additional fixation features extending from the base surface 1220. These additional fixation features can be any one of the fixation features such as posts, finned anchors, keels, etc. FIGS. 45C-45D show a glenoid implant embodiment 1200B that comprises such additional fixation features. The glenoid implant 1200B also has a peripheral ring 1212 structure as a peripheral fixation element similar to the glenoid implant 1200 and 1200A. The peripheral ring 1212 for the glenoid implant 1200B is the same structural features as the peripheral ring 1212 for the glenoid implants 1200 and 1200A including all of the optional features. The implant body 1205B is also shaped to mirror the outline of an anatomical glenoid. In the embodiment 1200B, however, the implant body 1205B comprises two or more stabilizing posts 925′ extending from a portion of the peripheral ring 1212 as the one or more additional fixation features mentioned above. The glenoid implant embodiments 1200, 1200A, and 1200B can be made of high modulus polymer material, such as UHMWPE or PEEK.

FIG. 45E is an illustration showing how a glenoid 24 may be prepared with an annular recess 24q for receiving the glenoid implants shown in FIGS. 44A and 45A. The annular recess 24q is dimensioned to receive the peripheral ring 1212 of the glenoid implants 1200, 1200A. To receive the implants 1200 or 1200A that have curved base surface 1220, the glenoid 24 is first reamed with a curved reamer to prepare the glenoid 24 surface to a curved surface 24s that matches the curvature of the curved base surface 1220. Then, a bell saw type reamer 225 is used to ream out the annular recess 24q.

FIG. 45F is an illustration showing the glenoid implant of FIG. 44A or 45A implanted in the glenoid 24 after the annular recess 24q has been formed.

FIGS. 45G-45H are illustrations showing examples of bell saw type reamers 225 and 226 that can be used to form the annular recess 24q in the glenoid 24.

In addition to the peripheral ring fixation feature, the glenoid implant can have one or more additional non-peripheral fixation features such as posts, finned anchors, or keels.

FIGS. 46A-46B are illustrations showing a glenoid implant example 1300 according to another embodiment. The glenoid implant 1300 comprises an implant body 1305 having an articulation surface 1330 on one side and a bone-facing base surface 1320 on opposing side. The glenoid implant 1300 further comprises a peripheral ring 1312 provided on the bone-facing base surface 1320 as a peripheral fixation element similar to the glenoid implants 1200 and 1200A. The glenoid implant 1300 also includes one or more posts 925″ extending from the base surface 1320 and located somewhere along the peripheral ring 1312 that can further enhance the implant's fixation with a bone. The

In some preferred embodiments, the one or more posts 925″ are located along the peripheral ring 1312 at radially symmetric positions. The radially spaced posts 925″ along with the peripheral ring 1312 are believed to minimize or substantially eliminate micromotion of the glenoid implant 1300 in a patient.

The peripheral ring 1312 and the posts 925″ are porous trabecular metallic structures that promote bone tissue ingrowth to enhance fixation of the glenoid implant 1300 to glenoid in a cement-less application.

Referring to the cross-sectional view in FIG. 46B, in some embodiments, the posts 925″ are a composite structure having a solid metal core that provides appropriate structural stability (i.e. rigidity) to the posts 925″. The solid metal core can be made of an appropriate alloy that allows the porous trabecular metal coating to bond to the solid metal core and provide lone lifetime of structural reliability in the patient. The combination of the peripheral ring fixation feature 1312 and the one or more additional fixation features 925″ having the solid metal core are referred to herein as having substantially formed of porous trabecular metallic material for promoting bone ingrowth when implanted into a patient.

The glenoid implant 1300 can be made of high modulus polymer material, such as UHMWPE or PEEK. In some embodiments, the implant body 1305 has a shape that mirrors the outline of an anatomical glenoid. The implant body 1305 of high modulus polymer material can be overmolded onto a portion of the metallic peripheral ring 1312 and post 925″ structure. The combination of the trabecular metallic peripheral ring 1312 and the one or more posts 925″ should provide enhanced primary fixation of the glenoid implant 1300 to a glenoid bone.

Although the devices, kits, systems, and methods have been described in terms of exemplary embodiments, they are not limited thereto. Rather, the appended claims should be construed broadly, to include other variants and embodiments of the devices, kits, systems, and methods, which may be made by those skilled in the art without departing from the scope and range of equivalents of the devices, kits, systems, and methods.

1. A glenoid implant comprising:
a main portion comprising:
a ring-shaped body defined by a hole extending therethrough, wherein the ring-shaped body comprises an articulation surface provided on one side configured to engage with a humeral head, and a base surface provided on an opposite side, configured to be secured into a glenoid.

2. The glenoid implant of claim 1, wherein the hole has a circular shaped outline.

3. The glenoid implant of claim 1, wherein the hole has a polygon shaped outline.

4. The glenoid implant of claim 1, wherein the hole has an irregular shaped outline.

5. The glenoid implant of claim 4, wherein the hole has a patient-specific irregular shaped outline.

6. The glenoid implant of claim 1, wherein the base surface is provided with one or more pegs for securing the implant in a glenoid.

7. The glenoid implant of claim 1, wherein the base surface can be concave, convex, or flat.

8. The glenoid implant of claim 6, wherein each of the one or more pegs comprises one or more slots for accommodating bone cement.

9. The glenoid implant of claim 1, wherein the articulating surface is formed of polyethylene such as ultra-high-molecular-weight polyethylene (UHMWPE), polyether ether ketone (PEEK), or hydrogel.

10. The glenoid implant of claim 1, wherein the main portion comprises at least a portion of its outer surface that is coated with a trabecular metallic material.

11. The glenoid implant of claim 1, wherein the main portion comprises a porous material portion including the base surface; and
a hydrogel portion that is bonded to the porous material portion and forming the articulation surface opposite from the base surface.

12.-71. (canceled)
